(19) United States
(12) Patent Application Publication
(10) Pub. No.: US 2022/0092774 A1
(43) Pub. Date: Mar. 24, 2022

(54) METHOD AND APPARATUS FOR ANALYSIS OF HISTOPATHOLOGY IMAGE DATA

(71) Applicant: Siemens Healthcare GmbH, Erlangen (DE)

(72) Inventors: Sven KOHLE, Erlangen (DE); Svenja LIPPOK, Uttenreuth (DE)

(73) Assignee: Siemens Healthcare GmbH, Erlangen (DE)

(21) Appl. No.: 17/474,142

(22) Filed: Sep. 14, 2021

(30) Foreign Application Priority Data
Sep. 22, 2020 (DE) 10 2020 211 843.4

(51) Int. Cl. G06T 7/00; G16H 10/60; G16H 15/00; G16H 30/40; G06N 3/04

(52) U.S. Cl. CPC G06T 7/0012; G16H 10/60; G16H 15/00; G06T 2207/20081; G06N 3/04; G06T 2207/30024; G16H 30/40

(57) ABSTRACT

Methods as well as systems are provided for analysis of histopathology image data. In an embodiment, a method, for provision of similarity information regarding different histopathology images of a patient, includes: provisioning first histopathology image data, based on a tissue sample taken from a patient at a first point in time; provisioning second histopathology image data, based on a tissue sample taken from the patient at a second point in time, different from the first point in time; analyzing, using an image processing algorithm, the first histopathology image data and the second histopathology image data for a similarity between at least one region from the first histopathology image data indicating a pathological appraisal and at least one region from the second histopathology image data indicating a pathological appraisal; determining similarity information based on the analyzing of the image processing algorithm for the similarity; and provisioning the similarity information determined.

### PRIORITY STATEMENT

The present application hereby claims priority under 35 U.S.C. § 119 to German patent application number DE102020211843.4 filed Sep. 22, 2020, the entire contents of which are hereby incorporated herein by reference.

### FIELD

Example embodiments of the invention generally relate to methods and apparatuses for analysis of histopathology image data. In particular embodiments of the present invention relate to a method and apparatus for analysis of different histopathology image data for similarities.

### BACKGROUND

The analysis of tissue samples with methods of histopathology is a central element in cancer diagnostics. In such methods tissue samples are taken from a patient from a region of the body in which there may possibly be a pathological change present. Typically a number of sections or blocks are obtained from a tissue sample, which are then cut into micrometer-thick tissue slides. In order to be able to better recognize or even just quantify tissue change, the tissue slides are stained with a histopathological staining. The analysis of these stained tissue slides under the microscope by a pathologist then allows information to be provided about possible pathological changes to the fine tissue structure of the tissue under examination.

Histopathological examinations are very labor-intensive. As well as the taking of the tissue samples as such, they require the preparation of the tissue slides, including the cutting, fixing and staining of the tissue slides. In such cases it should be noted that for each tissue sample a plurality of sections and tissue slides prepared therefrom must typically be analyzed.

To relieve the load on the medical personnel, these workflows have therefore in recent decades been ever more automated and digitized. Thus in modern laboratories computer-controlled automatic preparation and staining equipment is often already used. Moreover the stained tissue slides are mostly digitized nowadays for further use. Specialized scanners, known as whole slide scanners, are used for this. The image recorded by the scanners is also referred as a whole slide image. The histopathology image data obtained in this way is then viewed and analyzed by a pathologist at a digital diagnostic station.

While many processes of histopathological workflows can be improved and expedited by the advance of digitization, the appraisal as such—although carried out at a digital diagnostic station—is a situation in which the pathologist is still largely reliant on their own judgment. Moreover a histopathology image dataset for a tissue sample already represents a large volume of data. Thus the size of individual histopathology images is already comparatively extensive. The reason for this is that on the one hand histopathology images should be able to make possible an overview of the entire tissue slide, and on the other hand must have a sufficient resolution to make possible the observation of individual cells. Moreover a histopathology image dataset typically not only features an individual image for a tissue slide and a section but also for a number of individual images of different tissue slides from the tissue sample, which typically originate from different sections and have been stained with different histopathological stains. The appraisal is additionally rendered more difficult by the fact that histopathology image data is inherently inhomogeneous, which makes its quantification and reproducible classification much more complicated. Of similar complexity are the issues that must be addressed during the appraisal. As well as the basic question of whether a pathological change is present, a statement frequently also has to be made about whether tissue change is a new occurrence or whether it involves the recurrence of an anamnestically known disease.

All this and the implications of histopathological appraisals for the treatment of a patient has largely previously prevented an effective and full-coverage use of automated appraisal routines based on digitized image data. The consequence of this is that pathologists, because of the ongoing automation of the upstream processes, are confronted with an ever-greater workload, but for the actual appraisal are largely reliant on themselves. Because of the constantly growing volumes of data it is moreover increasingly difficult to include all available information and take it into account when assessing a case.

### SUMMARY

Embodiments of the present invention provide methods and apparatuses that support a user in the appraisal of digital histopathology image data.

In accordance with embodiments of the invention are directed to a method, an apparatus, a computer program product or a computer-readable memory medium. Advantageous developments are specified in the claims.

In accordance with one form of embodiment of the invention, a computer-implemented method for provision of similarity information regarding different histopathology image data of a patient is provided. The method has a number of steps. One step is directed to the provision of first histopathology image data. The first histopathology image data is based on a tissue sample that has been taken from a patient at a first point in time. A further step is directed to the provision of second histopathology image data. The second histopathology image data is based on a tissue sample that has been taken from the patient at a second point in time different from the first. A further step is directed to the determination of similarity information with an image processing algorithm based on the first and second histopathology image data. The similarity information has a specification regarding a similarity between at least one region in the first histopathology image data indicating a pathological appraisal and at least one region in the second histopathology image data indicating a pathological appraisal. A further step is directed to the provision of the similarity information.

In accordance with a further form of embodiment a computer-implemented method for provision of similarity information regarding different histopathology image data of a patient is provided. The method has a number of steps. One step is directed to a provision of first histopathology image data, which is based on a tissue sample that has been taken from a patient at a first point in time. A further step is directed to a provision of second histopathology image data, which is based on a tissue sample that has been taken from a patient at a second point in time different from the first point in time. A further step is directed to an identification of a region of interest in the first histopathology image data. A further step is directed to searching through the second histopathology image data for regions of similarity, with the regions of similarity each having a similarity with the region of interest. In this case the step of searching features the application of the image processing algorithm to the second histopathology image data. A further step is directed to determining similarity information based on the step of searching. A further step is directed to provision of the similarity information.

In accordance with one form of embodiment a system for provision of similarity information regarding different histopathology image data of a patient is provided. The system has an interface and a controller. The interface is embodied for receiving first histopathology image data and second histopathology image data, wherein the first histopathology image data is based on a tissue sample that was taken from a patient at a first point in time, and the second histopathology image data is based on a tissue sample that was taken from the patient at a second point in time different the first point in time. The computing unit is embodied, based on the first and second histopathology image data, to determine similarity information with an image processing algorithm, with the similarity information having a specification of a similarity between at least one region from the first histopathology image data indicating a pathological appraisal and at least one region from the second histopathology image data indicating a pathological appraisal. The computing unit is further embodied to provide the similarity information.

In accordance with one form of embodiment, the system further has a database for storage of a number of items of histopathology image data and a user interface for interaction with a user. The interface has a data connection to the database and to the user interface. The controller is further embodied to select the first histopathology image data from the database based on a manual entry of the user in the user interface and to receive it via the interface. The controller is further embodied to select the second histopathology image data from the database based on the first histopathology image data and/or based on metadata assigned to the first histopathology image data.

In accordance with a further form of embodiment, a computer program product comprises a program and is able to be loaded directly into a memory of a programmable controller and has program code/segments, e.g. libraries and auxiliary functions for carrying out a method for provision of similarity information, in particular in accordance with the aforementioned forms of embodiment, when the computer program product is executed.

In accordance with a further form of embodiment, a computer-readable memory medium is disclosed, on which readable and executable program sections are stored for carrying out all steps of a method for providing similarity information in accordance with the aforementioned forms of embodiment, when the program sections are executed by the controller.

In accordance with a further form of embodiment, a computer-implemented method, for provision of similarity information regarding different histopathology image data of a patient, comprises:

provisioning first histopathology image data, based on a tissue sample taken from a patient at a first point in time;

provisioning second histopathology image data, based on a tissue sample taken from the patient at a second point in time, different from the first point in time;

analyzing, using an image processing algorithm, the first histopathology image data and the second histopathology image data for a similarity between at least one region from the first histopathology image data indicating a pathological appraisal and at least one region from the second histopathology image data indicating a pathological appraisal;

determining similarity information based on the analyzing of the image processing algorithm for the similarity; and

provisioning the similarity information determined.

In accordance with a further form of embodiment, a system for provision of similarity information regarding different histopathology image data of a patient, comprises:

an interface embodied to receive first histopathology image data and second histopathology image data, the first histopathology image data being based on a tissue sample taken from a patient at a first point in time, and the second histopathology image data being based on a tissue sample taken from the patient at a second point in time, different from the first; and

a computing device embodied to:
determine, based on the first histopathology image data and second histopathology image data, similarity information using an image processing algorithm, the similarity information including a specification of similarity between at least one region from the first histopathology image data indicating a pathological appraisal and at least one region from the second histopathology image data indicating a pathological appraisal; and
provide the similarity information determined.

In accordance with a further form of embodiment, a non-transitory computer program product stores a program, directly loadable into a memory of a programmable computing device a controller, including program code for carrying out the method of an embodiment when the program is executed in the controller.

In accordance with a further form of embodiment, a non-transitory computer-readable memory medium stores readable and executable program sections for executing the method of claim 1 when the program sections are executed by a controller.

### BRIEF DESCRIPTION OF THE DRAWINGS

Further special aspects and advantages of the invention are evident from the explanations of example embodiments given below with the aid of schematic drawings. Modifications given in this context can each be combined with one another in order to embody new forms of embodiment. In different figures the same reference characters are used for the same features.

In the figures:

FIG. 1 shows a schematic diagram of a form of embodiment of a system for provision of similarity information based on histopathology image data,

FIG. 2 shows a flow diagram of a method for providing similarity information based on histopathology image data in accordance with a form of embodiment,

FIG. 3 shows a flow diagram of a method for providing similarity information based on histopathology image data in accordance with a further form of embodiment,

FIG. 4 shows a schematic diagram of regions of interest extracted from histopathology image data in accordance with a form of embodiment,

FIG. 5 shows a schematic diagram of regions of interest extracted from histopathology image data in accordance with a further form of embodiment,

FIG. 6 shows a schematic diagram of regions of similarity determined in histopathology image data in accordance with a form of embodiment,

FIG. 7 shows a flow diagram of a method for providing similarity information based on histopathology image data in accordance with a further form of embodiment,

FIG. 8 shows a flow diagram of a method for determining regions of similarity in histopathology image data in accordance with a form of embodiment,

FIG. 9 shows a schematic diagram of a form of embodiment of an image processing algorithm, which is embodied to provide similarity information based on histopathology image data,

FIG. 10 shows a schematic diagram of a further form of embodiment of an image processing algorithm, which is embodied to provide similarity information based on histopathology image data,

FIG. 11 shows a schematic diagram of a further form of embodiment of an image processing algorithm, which is embodied to provide similarity information based on histopathology image data, and

FIG. 12 shows a flow diagram of a method for providing similarity information based on histopathology image data in accordance with a further form of embodiment.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The inventive embodiments are described below both with regard to the apparatuses and also with regard to the methods. Features, advantages or alternate forms of embodiment mentioned here are likewise to be transferred to the other claimed subject matter and vice versa. In other words the physical claims (which are directed to an apparatus for example) can also be developed with the features that are described or claimed in conjunction with a method. The corresponding functional features of the method are embodied in such cases by corresponding physical modules.

The inventive embodiments are furthermore described both with regard to methods and apparatuses for visualization of a three-dimensional body and also with regard to methods and apparatuses for adaptation of trained functions. Features and alternate forms of embodiment of data structures and/or functions for methods and apparatuses for determination can be transferred here to similar data structures and/or functions for methods and apparatuses for adaptation. Similar data structures here can be identified in particular by the use of the prefix “training”. Furthermore the trained functions used in methods and apparatuses for analysis of histopathology image data can have been adapted and/or can have been provided by methods and apparatuses for adaptation of trained functions.

In accordance with one form of embodiment of the invention, a computer-implemented method for provision of similarity information regarding different histopathology image data of a patient is provided. The method has a number of steps. One step is directed to the provision of first histopathology image data. The first histopathology image data is based on a tissue sample that has been taken from a patient at a first point in time. A further step is directed to the provision of second histopathology image data. The second histopathology image data is based on a tissue sample that has been taken from the patient at a second point in time different from the first. A further step is directed to the determination of similarity information with an image processing algorithm based on the first and second histopathology image data. The similarity information has a specification regarding a similarity between at least one region in the first histopathology image data indicating a pathological appraisal and at least one region in the second histopathology image data indicating a pathological appraisal. A further step is directed to the provision of the similarity information.

First and second histopathology image data are image datasets, which can have one or more especially two-dimensional individual images. Another expression for the first histopathology image data is first histopathology image dataset. Another expression for the second histopathology image data is second histopathology image dataset. The individual image or the individual images can each be pixel images. The individual image or the individual images each map a tissue slide that has been prepared from a tissue sample of the patient. If a number of tissue slides are mapped in a histopathology image dataset, all these tissue slides can have been prepared from the same tissue sample. All image data in the first histopathology image data can thus have been created based upon one tissue sample and all image data in the second histopathology image data can have been created based upon another/a further tissue sample. The two tissue samples can both have been taken from the same patient, and indeed especially from the same or at least one similar anatomical target region of the patient, but at different points in time however. Days, months or years as well as diverse medical treatments of the patient can lie between the points in time for example.

The preparation of the tissue slides from the tissue samples can comprise the preparation of a section from the tissue sample (for example with a punch tool), with the section being cut into micrometer-thick slices, the tissue slides. Another word for section is block or punch biopsy. The tissue slides mapped in histopathology image data can in this case in particular have been obtained from different sections from the same tissue sample. Under microscopic observation the individual images of the histopathology image data can show the fine tissue structure of the tissue sample and in particular the cell structure or the cells contained in the tissue sample. When observed on a greater length scale the individual images can show an overview of the tissue structure and tissue density.

The preparation of the tissue slides further comprises the staining of the tissue slides with a histopathological staining. The staining in this case can serve to highlight different structures in the tissue slide, such as e.g. cell walls or cell nuclei, or to test a medical indication, such as e.g. a cell proliferation level. Different histopathological stains are used for different purposes in such cases. In particular all individual images contained in a histopathology image dataset can map tissue slides that have been stained with the same histopathological staining. As an alternative the individual images contained in a histopathology image dataset cam map tissue slides that have been stained with different histopathological stains.

To create the histopathology image data the stained tissue slides are digitized or scanned. To this end the tissue slides are scanned with a suitable digitizing station, such as for example a whole slide scanner, which preferably scans the entire tissue slide mounted on an object carrier and converts it into a pixel image. In order to preserve the color effect from the histopathological staining, the pixel images are preferably color pixel images. Since in the appraisal both the overall impression of the tissue and also the finely resolved cell structure is of significance, the individual images contained in the histopathology image data typically have a very high pixel resolution. The data size of an individual image can typically amount to several gigabytes. The digitized recordings of the tissue slides can where necessary be grouped together to form a histopathology image dataset. As an alternative an individual recording can also form the histopathology image data. The histopathology image data can be processed digitally and especially archived in a suitable database.

As well as image data the histopathology image data can also contain metadata, in which for example the point in time at which the tissue sample was taken, a patient identifier, one or more histopathological stains used, a pathological finding and/or an anatomical target region, from which the tissue sample originates, can be stored. As an alternative or in addition such information can be stored in the database archiving the histopathology image data or in a database separate therefrom. Such databases can for example be part of one or more medical information systems, such as for example Hospital Information Systems (HIS), Radiology Information Systems (RIS), Laboratory Information Systems (LIS), Cardiovascular Information Systems (CVIS) and/or Picture Archiving and Communicating Systems (PACS).

The expression “based on a tissue sample” can therefore mean overall that the respective histopathology image data image has data, which shows tissue slides that were prepared from the tissue sample and have been stained with a histopathological staining.

‘Provision’ with regard to the histopathology image data can mean that the data is made available from a digitization station for further use. Provision can furthermore mean that the data is or will be able to be retrieved from a corresponding database, and/or is loaded or is able to be loaded into a computing unit in order for the histopathology image data to undergo one or more processing steps, e.g. in a data processing facility.

The image processing algorithm can in particular be construed as a computer program product, which is embodied to determine similarity information through analysis of image data or pixel values of the first and second histopathology image data. The image processing algorithm can have program elements in the form of one or more instructions for a processor to determine the similarity information. The image processing algorithm can be provided for example by being stored in a memory facility or being loaded into a working memory of a suitable data processing facility or by generally being made available for use.

A region indicating a pathological appraisal can in particular show or suggest one or more pathological changes to the imaged tissue. In other words a region indicating a pathological appraisal can be a region indicating one or more pathological changes. For example a region indicating a pathological appraisal can feature one or more tumor cells or one and/or more diseased tissue structures. The region indicating a pathological appraisal can be identified automatically in the first and second histopathology image data in each case and/or be identified by a user. The user can be a doctor or a pathologist for example.

A similarity between regions indicating a pathological appraisal can in particular be a morphological or structural similarity of the regions in question. For example similar regions can have a similar tissue structure, a similar texture, similar pixels or stain values, a similar cell density, a similar cell morphology, similar patterns and/or further similar features. To this end the image processing algorithm can be embodied to extract such and further features automatically from the first and second histopathology image data and compare them between the first and second histopathology image data, in order to determine a quantitative measure for the similarity (measure of similarity) therefrom.

The similarity information is created based on the similarity analysis. In particular the similarity information can have an indication of the similarity between the regions indicating a pathological appraisal. Furthermore the similarity information can specify a similarity between the regions indicating a pathological appraisal. In particular the similarity information can specify a quantitative measure for the similarity (measure of similarity) or can be based on such a measure.

The provision of the similarity information can comprise a provision of the similarity information for any given further use. For example the similarity information can be provided to a further algorithm for further analysis. The similarity information can further be provided for archiving in a database. Furthermore the similarity information can be provided via a user interface.

Through the provision of similarity information a statement is made available as to the extent to which pathological changes in the first and second histopathology image data are similar. Thus possible similarities are not sought in an unspecific way in the overall histopathology image data, but specifically for those regions that show a pathological tissue change or point to such a change. The inventors have recognized that such information can be relevant in particular for the question of whether a tissue change has newly occurred, i.e. whether a new disease is involved, or whether a reinflammation or re-spreading of an anamnestically already known disease. Such a phenomenon is also referred to as recidive. It is namely shown that tissue changes that date back to a reinflammation or re-spreading of a basic disease have similar morphological and/or structural features. The automated evaluation of these similarities not only makes it possible to discover subtle or hidden similarities, which can remain hidden from the human eye, but also guarantees a rapid, systematic and exhaustive comparison of the available image data. The latter above all is often not possible for a user without support in view of the enormous image sizes and amounts of data. The user thus has valuable additional information to hand when for example it is a matter of deciding whether for example a therapy concept was successful or has to be adapted. Based on the identification of a medically relevant parameter and its automated evaluation in digitized measurement data, the inventors have thus created a method that gives the user sustained support in the making of a medical diagnosis.

In accordance with one form of embodiment the method can further have a step of identification of regions indicating a pathological appraisal in the first and/or second histopathology image data.

In other words regions relevant for the above issue, i.e. those regions in the histopathology image data that show or indicate a pathological tissue change, are determined automatically or semi-automatically. In this case the regions can be identified in both the first histopathology image data and also in the second histopathology image data or only in one of the two. For the identification there can be recourse in this case to a user's annotations that already exist, which the user has set up for example during a previous appraisal. To this end for example the metadata of the corresponding histopathology image data can be evaluated. As an alternative or in addition the entire histopathology image data can be re-analyzed in each case. The image processing algorithm can be embodied accordingly for this for example, so that the identification of the regions indicating a pathological appraisal can be undertaken by applying the image processing algorithm to the first and/or second histopathology image data. As a further alternative a user entry relating to regions indicating a pathological appraisal can be evaluated. For example the user can mark one or more regions via a user interface, which are then to be used as a basis for further processing as further regions (also called regions of interest below).

The identification of regions indicating a pathological appraisal enables similarities between pathological tissue changes to be explicitly searched for. As a result a corresponding statement can be provided within a short time and with high confidence. Moreover the user's task, that of creating a medical diagnosis, can be further made easier by an at least semi-automated selection of relevant regions.

In accordance with one form of embodiment the similarity information comprises:

a specification of similar regions indicating a pathological funding in the respective first and/or second histopathology image data;

an assistance image based on the first and/or second histopathology image data, in which similar regions indicating a pathological appraisal are highlighted;

location information about similar regions indicating a pathological appraisal in the first and/or second histopathology image data;

a quantitative specification of a similarity between similar regions indicating a pathological appraisal in the respective first and second histopathology image data;

a specification as to whether a recidive relationship exists between the first and second histopathology image data.

Relevant information is provided to the user by the the similarity information for the analysis and appraisal of histopathology image data. Through the specification of the regions with similar pathological changes the user is explicitly alerted to regions, which could point to the recurrence of a disease for example. With the aid of the similarity regions they can make their own image of a possible similarity between pathological changes in consecutive tissue samples. The regions in such cases can be characterized by location information, with the aid of which the regions can be drawn-in in a graphical representation in the first and/or second histopathology image data, for example. The location information in this case can comprise a specification of coordinates.

As well as this, in accordance with a few forms of embodiment information about a relative proportion of regions indicating a pathological appraisal in the respective histopathology image data can be provided. In this way the user is given information about how pathological tissue changes have developed over time.

As well as this, one or more assistance images can be provided. The assistance images can be based on image data of the first and/or second histopathology image data or be rendered based upon this image data. The regions indicating a pathological appraisal can be indicated by a marking, for example, such as in the form of a frame or a mask, and/or highlighted in color in the assistance images.

In such cases, in the assistance images, in particular each, especially all regions inducing a pathological appraisal and/or those regions inducing a pathological appraisal can be identified, which have similarity beyond the histopathology image data. Thus, on the one hand, the user is given an overview of all tissue changes and on the other hand is given an indication of areas that point to a recidive. For example regions inducing a pathological appraisal can generally be identified by one color and similar regions by another color.

As an alternative or in addition, through the optional quantitative specification of a similarity, a user is given a statement about the degree of similarity. They can thereby decide which regions have a great similarity and concentrate on these in their analysis. The quantitative specification can be provided as a numerical value for example or be integrated into the assistance image (for example as a numerical specification or in the form of color coding).

The similarity information can further include a specification about a recidive relationship between the first histopathology image data and the second histopathology image data. In other words this is a specification about whether pathological changes, which are visible in either the first histopathology image data or the second histopathology image data, are recognizable in a similar way in the other histopathology image data in each case. This thus represents a statement about whether a pathological change is based on a re-inflammation or a further growth of a pathological change already present. Following an idea of the invention, such a statement can be made based on the quantitative specification or specifications of a similarity. The average value or median or a maximum value of the quantitative specifications of a similarity can be evaluated for this for example.

In accordance with one form of embodiment the step of providing the similarity information features a display of the similarity information for a user via a user interface, by which the user can immediately be made aware of the result of the similarity analysis.

In accordance with one form of embodiment the method further has the step of filling out a medical report template based on the similarity information.

The automated filling out of a report template enables the load on the user to be relieved further in the appraisal of histopathology image data. The report template can be an electronic medical report for example. Placeholders can be provided in the report template for entry of case-specific information. One or more placeholders can be filled out in the filling-out step based on the similarity information.

In accordance with one form of embodiment the step of analyzing comprises an identification of a region of interest indicating a pathological appraisal in the first histopathology image data and also a search through the second histopathology image data for regions of similarity, with the regions of similarity each having a similarity with the region of interest. In this case the step of searching comprises the application of the image processing algorithm to the second histopathology image data and the step of determining the similarity information based on the step of searching.

In other words the region of interest represents one or more regions of the first histopathology image data indicating a pathological appraisal (or a pathological change). The region of interest can in particular already be predetermined (such as by an earlier appraisal) or be dynamically predetermined (such as by a user entry or automatically). Depending on the region of interest, the regions of similarity are conversely determined in the second histopathology image data. The regions of similarity can be construed as regions of the second histopathology image data indicating a pathological appraisal (or a pathological change). A similarity between the regions of interest and the regions of similarity can again comprise a morphological and/or structural similarity. For example similar regions can have a similar tissue structure, a similar texture, similar pixel or color values, a similar cell density, a similar cell morphology, similar patterns and/or further similar features. The image processing algorithm can be embodied automatically to extract and to compare such and further features automatically from the region of interest and possible regions of similarity. The image processing algorithm can further be embodied, based on the comparison, to determine a quantitative measure for the similarity (measure of similarity). Regions of similarity can then in particular be those regions for which the measure of similarity lies above a predetermined or predeterminable threshold. Such a threshold in this case can be determined automatically or be predetermined by a user. The threshold can further be determined semi-automatically by a threshold being proposed to a user.

Through the identification of the regions of interest in the first histopathology image data a search can be made specifically in the second dataset for regions similar thereto. This enables morphological and/or structural similarities of pathological changes to be discovered and revealed in a targeted manner in tissue samples of the patient taken at different times. This puts the user in a position to make a well-founded statement about whether for example pathological change in a newly taken tissue sample is a recidive of an already known pathological change. This enables the user to be supported effectively in the appraisal and above all in making a diagnosis or prognosis.

In accordance with one form of embodiment the region of interest has one or more individual regions defined in the first histopathology image data.

The individual regions in this case can each indicate one or more pathological appraisals or show one or more pathological tissue changes. The individual regions can for example have extracts from the first histopathology image data and thus likewise have (pixel) image data. Thus the region of interest can likewise have (pixel) image data. In particular one or more of the regions defined in the histopathology image data can also comprise a (complete) individual image of the first histopathology image data or the entire histopathology image data. Accordingly the region of interest can also comprise one or more individual images of the first histopathology image data or the entire histopathology image data. The regions defined in the first histopathology image data can have different shapes. For example the defined regions can be rectangular or circular or have any other given delimitation. Taking into consideration a number of regions for the region of interest enables a larger base of data to be provided for the search for similar regions. Conversely the region of interest can be restricted to just one region or extract in the first histopathology image data, if only this appears relevant. Overall, by the adaptive definition of the region of interest, a good match to the respective circumstances is made possible.

In accordance with one form of embodiment the step of identification of the region of interest comprises a determination of the region of interest by the image processing algorithm, and/or an evaluation of an annotation of a users, with the annotation identifying the region of interest. In this case the annotation can be provided in particular after the step of provision of the first histopathology image data by a manual entry of a user via a user interface.

In other words the region of interest can be determined automatically and/or manually by a user. This enables the region of interest to be defined case-specifically and flexibly. In particular the annotation of the user can identify one or more regions in the first histopathology image data. To this end the user can be shown one or more reference images of the first histopathology image data in a user interface. A reference image can be a presentation created based upon the respective histopathology image data for display via a user interface. An annotation of a user by a user entry can be set up for example by pointing to a relevant region of a reference image, by drawing a frame around a relevant region in the reference image, and/or by circling a relevant region in the reference image. This can be done for example with a mouse or with an electronic stylus or by gesture control. One or more annotations, e.g. from an earlier appraisal, can further be present. These can be stored as metadata for the first histopathology image data (for example in the first histopathology image data itself or in a separate database). By evaluating these annotations already set up, the scan be used for the definition of the region of interest. In it is also possible, based on an annotation of a user, to identify further relevant regions in the first histopathology image data automatically and assign them to the region of interest. In such cases data can be searched for in the first histopathology image data according to regions that exhibit a similarity to the regions characterized by the annotation of the user. Accordingly the image processing algorithm can be embodied to search automatically in histopathology image data for regions indicating a pathological appraisal and in doing so, where necessary to take into account regions selected beforehand by an annotation of a user.

In accordance with one form of embodiment the step of searching comprises an extraction of a feature signature based on the region of interest and a determination of the similarity information based on the extracted feature signature.

The feature signature can have one or more features extracted from the region of interest and in particular from the image data of the region of interest or have been computed from these. As well as this the feature signature, based on (or by taking additional account of) further information, such as e.g. a surrounding region around the region of interest, can be extracted from the entire first histopathology image data and/or metadata for the first histopathology image data. The feature signature can in particular characterize the region of interest. The features of the feature signature can be grouped together into a feature vector. In particular the feature signature can have such a feature vector. The features can be morphological and/or structural features and/or features relating to a texture and/or a pattern. In particular the features can comprise a tissue structure or a tissue density. The features can further feature a cell density, a cell morphology, a distribution of a histopathological staining, a cell size, a distribution of one ore more specific cell class(es) and the like.

The image processing algorithm can further be embodied to establish the similarity information based on the feature signature.

The establishment of the similarity information can comprise a determination of possible regions of similarity in the second histopathology image data. The establishment of the similarity information can further comprise the extraction of a feature signature in each case from the possible regions of similarity. In this case the process can be the same as the extracted feature signature based on the region of interest. The establishment of the similarity information can further comprise a comparison of the feature signature based on the possible regions of similarity in each case with the feature signature extracted based on the region of interest. The establishment of the similarity information can further comprise a measure of similarity based on the comparison in each case for the possible regions of similarity and the establishment of the similarity information based on the measure or measures of similarity.

The step of comparison can in particular be based on the determination of a distance between the respective feature signatures, the computation of a cosine similarity of the feature signatures and/or the computation of a weighted sum of the difference or the similarity between individual features of the feature signatures. Those regions of the second histopathology image data of which the associated measure of similarity is greater than a predetermined or predeterminable threshold can be identified as regions of similarity.

The use of feature signatures enables simple-to-implement and easily transferrable parameters for a reconciliation of different image data to be defined. Moreover the features contained in the feature signatures can be based on higher-ranking observables derived from the image data, which often better characterize the properties of the mapped structures than the underlying image data itself.

In accordance with one form of embodiment the image processing algorithm has one or more trained functions.

A trained function generally maps input data to output data. The output data can in particular furthermore depend on one or more parameters of the trained function. The one or more parameters of the trained function can be determined and/or adapted by training. The determination and/or the adaptation of the one parameter or the number of parameters of the trained function can be based in particular on a pair consisting of training input data and associated training output data, wherein the trained function can be applied to the training input data to create training mapping data. In particular the determination and/or the adaptation can be based on a comparison of the training mapping data and the training output data. In general a trainable function, i.e. a function with not yet adapted parameters, is referred to as a trained function. By training one or more trainable functions optionally contained in the image processing algorithm, the image processing algorithm can be embodied to carry out one or more tasks described in conjunction with the image processing algorithm, such as e.g. the analyzing of the first histopathology image data and of the second histopathology image data for a similarity between at least one region from the first histopathology image data indicating a pathological appraisal and at least one region from the second histopathology image data indicating a pathological appraisal, the searching of the second histopathology image data for regions of similarity, the identifying of a region of interest in the first histopathology image data indicating a pathological appraisal, the determination of similarity information, the extraction of a feature signature and/or the establishing of the regions of similarity or of the similarity information based on the extracted feature signature. If a number of these tasks are realized by a trained function, the image processing algorithm can have a separate trained function for each of these tasks. As an alternative or in addition a trained function can be embodied or trained to handle a number of these tasks through to all tasks.

Other terms for trained function are trained mapping specification, mapping specification with trained parameters, function with trained parameters, algorithm based on artificial intelligence, machine-learning algorithm. An example of a trained function is an artificial neural network. Instead of the term “neural network” the term “neural net” can also be used.

A neural network is fundamentally structured like a biological neural network—such as a human brain. In particular an artificial neural network comprises an input layer and an output layer. It can further comprise a number of layers between input and output layer. Each layer comprises at least one, preferably a number of, nodes. Each node can be understood as a biological processing unit, e.g. as a neuron. In other words each neuron corresponds to an operation that is applied to input data. Nodes of a layer can be connected to other nodes of other layers by edges, in particular by directed edges or connections. These edges or connections define the flow of data between the nodes of the network. The edges or connections are associated with a parameter that is frequently referred to as “weight” or “edge weight”. This parameter can regulate the importance of the output of a first node for the input of a second node, wherein the first node and the second node are connected by an edge. In particular a trained function can also have a deep neural network or deep artificial neural network.

In particular a neural network can be trained. In particular the training of a neural network is carried out based on the training input data and the associated training output data in accordance with a supervised learning technique, wherein the known training input data is entered into the neural network and the output data generated by the network is compared with the associated training output data. The artificial neural network learns and adapts the edge weights for the individual nodes independently for as long as the output data of the last network layer does not sufficiently correspond to the training output data.

In accordance with one form of embodiment at least one of the trained functions has a convolutional neural network and in particular a region-based convolutional neural network.

In particular the convolutional neural network can be embodied as a deep convolutional neural network. The neural network has one or more convolutional layers and one or more deconvolutional layers. In particular the neural network can have a pooling layer. The use of convolutional layers and/or deconvolutional layers enables neural networks to be employed especially efficiently for image processing, since despite many connections between node layers, only few edge weights (namely the edge weights corresponding to the values of the convolutional kernel) have to be determined. Thus, for the same number of training data items, the accuracy of the neural network can also be improved.

The region-based convolutional neural network can have a fast region-based convolutional neural network”) or a fast region-based convolutional neural network. Region-based convolutional neural networks are characterized by having integrated functionalities for definition of possibly relevant image regions, which makes them suitable for a region-by-region determination of similarities in accordance with forms of embodiment of the invention.

In accordance with one form of embodiment the method further comprises a step of receiving an acknowledgement of the user relating to the similarity information via a user interface and also a step of adapting the trained function, whereby the function can be continuously improved during use (known as continuous learning).

In accordance with one form of embodiment the second point in time lies before the first point in time. In other words the first histopathology image data thus involves data of a follow-up examination. This enables the user to define a region of interest in the first histopathology image data for example and regions of similarity are searched for automatically in the second histopathology image data and provided to the user. On this basis the user can then for example decide whether a pathological change in the region of interest marked by them is a recidive of a pathological change, which was already visible in the second histopathology image data.

In accordance with one form of embodiment the step of providing the second histopathology image data comprises accessing a database for histopathology image data and selecting the second histopathology image data from the histopathology image data stored in the database based on the first histopathology image data and/or on metadata assigned to the first histopathology image data. In accordance with forms of embodiment the selection can be based, as an alternative or in addition, on metadata assigned to the second histopathology image data.

In accordance with one form of embodiment the metadata can have:

a patient identifier for identification of the patient,

information about an anatomical target region of the patient from which the tissue sample was taken, on which the first histopathology image data is based,

information about an anatomical target region of the patient from which the tissue sample was taken, on which the second histopathology image data is based,

information in respect of the histopathological staining or stains used in the provision of the first histopathology image data,

information in respect of the histopathological staining or stains used in the provision of the second histopathology image data,

a suspected diagnosis or a suspected appraisal based on the first histopathology image data,

a diagnosis or an appraisal based on the second histopathology image data,

information regarding the second point in time, and/or

information regarding the first point in time.

This not only enables the second histopathology image data to be found automatically, but also enables especially suitable second histopathology image data to be provided. Through this the load on the user can be further relieved. In this case the provision can further be based on the metadata assigned to the second histopathology image data and in particular based on a comparison of the metadata assigned to the first histopathology image data with the metadata assigned to the second histopathology image data. The suspected diagnosis or the suspected appraisal in this case can in particular be input by the user via the user interface.

In accordance with one form of embodiment the step of provision of the first histopathology image data comprises a selection of the first histopathology image data by a user via a user interface. This enables the user explicitly to select the first histopathology image data that they wish to process.

In accordance with one form of embodiment the first point in time lies before the second point in time. In other words the second histopathology image data thus involves data of a follow-up examination. Thus the user does not have to first define region of interest in histopathology image data for example, but known regions of in the—in this form of embodiment “old”—first histopathology image data are also used. On this basis regions of similarity in the second histopathology image data are then automatically sought and provided.

In accordance with one form of embodiment the step of provision of the first histopathology image data comprises accessing a database for histopathology image data and selecting the first histopathology image data from the histopathology image data stored in the database based on the second histopathology image data and/or metadata assigned to the second histopathology image data.

In accordance with one form of embodiment the metadata can have:

information about an anatomical target region of the patient from which the tissue sample has been taken, on which the first histopathology image data is based,

information about an anatomical target region of the patient from which the tissue sample has been taken, on which the second histopathology image data is based,

information in respect of the histopathological staining or stains used in the provision of the first histopathology image data,

information in respect of the histopathological staining or stains used in the provision of the second histopathology image data,

a suspected diagnosis or a suspected appraisal based on the second histopathology image data,

a diagnosis or an appraisal based on the first histopathology image data,

information regarding the second point in time, and/or

information regarding the first point in time.

This enables the first histopathology image data not only to be found automatically but also enables especially suitable first histopathology image data to be provided. This enables the load on the user to be further relieved. In this case the provision can further be based on the metadata assigned to the first histopathology image data and in particular based on a comparison between the metadata assigned to the first histopathology image data and the metadata assigned to the second histopathology image data. By taking into account a suspected diagnosis or a suspected appraisal, the first histopathology image data can be found in a targeted manner, since for example an explicit search can be made for second histopathology image data that has a similar diagnosis or a similar appraisal. The suspected diagnosis or the suspected appraisal can be entered by the user via a user interface in accordance with forms of embodiment of the invention.

In accordance with one form of embodiment the step of provision of the second histopathology image data comprises the selection of the second histopathology image data by a user via a user interface. This enables the user explicitly to select the histopathology image data that he wishes to process.

In accordance with one form of embodiment the tissue sample on which the first histopathology image data is based and the tissue sample on which the second histopathology image data is based have each been taken from the same or from at least one similar anatomical target region of the patient. The same anatomical target region can mean for example that the tissue sample has been taken from the same organ or the same anatomy or the same tissue region of the patient. The same anatomical target region can further mean that the respective removal points of tissue samples relative to the patient have approximately the same coordinates.

In accordance with a further form of embodiment, a computer-implemented method for provision of similarity information regarding different histopathology image data of a patient is provided. The method has a number of steps. One step is directed to a provision of first histopathology image data, which is based on a tissue sample that has been taken from a patient at a first point in time. A further step is directed to a provision of second histopathology image data, which is based on a tissue sample that has been taken from a patient at a second point in time different from the first point in time. A further step is directed to an identification of a region of interest in the first histopathology image data. A further step is directed to searching through the second histopathology image data for regions of similarity, with the regions of similarity each having a similarity with the region of interest. In this case the step of searching features the application of the image processing algorithm to the second histopathology image data. A further step is directed to determining similarity information based on the step of searching. A further step is directed to provision of the similarity information.

In accordance with one form of embodiment, a system for provision of similarity information regarding different histopathology image data of a patient is provided. The system has an interface and a controller. The interface is embodied for receiving first histopathology image data and second histopathology image data, wherein the first histopathology image data is based on a tissue sample that was taken from a patient at a first point in time, and the second histopathology image data is based on a tissue sample that was taken from the patient at a second point in time different the first point in time. The computing unit is embodied, based on the first and second histopathology image data, to determine similarity information with an image processing algorithm, with the similarity information having a specification of a similarity between at least one region from the first histopathology image data indicating a pathological appraisal and at least one region from the second histopathology image data indicating a pathological appraisal. The computing unit is further embodied to provide the similarity information.

The controller can be embodied as a central or local computing unit. The computing unit can have one or more processors. The processors can be embodied as a central processing unit (abbreviated to CPU) and/or as a graphics processing unit (abbreviated to GPU). As an alternative the controller can be implemented as a local or cloud-based processing server.

The interface can generally be embodied for the exchange of data between the controller and further components. The interface can be implemented in the form of one or more individual data interfaces, which can have a hardware and/or software interface, e.g. a PCI bus, a USB interface, a Firewire interface, a ZigBee or a Bluetooth interface. The interface can further feature an interface of a communication network, wherein the communication network can feature a Local Area Network (LAN), for example an Intranet or a Wide Area Network (WAN). Accordingly the one or more data interfaces can have a LAN interface or a Wireless LAN interface (WLAN or Wi-Fi).

The advantages of the proposed system essentially correspond to the advantages of the proposed methods. Features, advantages or alternate forms of embodiment can likewise be transferred to the other claimed subject matter and vice versa.

In accordance with one form of embodiment, the system further has a database for storage of a number of items of histopathology image data and a user interface for interaction with a user. The interface has a data connection to the database and to the user interface. The controller is further embodied to select the first histopathology image data from the database based on a manual entry of the user in the user interface and to receive it via the interface. The controller is further embodied to select the second histopathology image data from the database based on the first histopathology image data and/or based on metadata assigned to the first histopathology image data.

In accordance with a further form of embodiment, a computer program product comprises a program and is able to be loaded directly into a memory of a programmable controller and has program code/segments, e.g. libraries and auxiliary functions for carrying out a method for provision of similarity information, in particular in accordance with the aforementioned forms of embodiment, when the computer program product is executed.

In accordance with a further form of embodiment, a computer-readable memory medium is disclosed, on which readable and executable program sections are stored for carrying out all steps of a method for providing similarity information in accordance with the aforementioned forms of embodiment, when the program sections are executed by the controller.

The computer program products in this case can comprise software with a source code that still has to be compiled and linked or only has to be interpreted, or an executable software code, which for execution only has to be loaded into the processing unit. The computer program products enable the methods to be carried out in a quick, identically repeatable and robust manner. The computer program products are configured so that the computing units can carry out the inventive method steps of an embodiment. The computing unit in such cases must have the respective prerequisites such as for example a corresponding main memory, a corresponding processor, a corresponding graphics card or a corresponding logic unit, so that the respective method steps can be carried out efficiently.

The computer program products are stored for example on a computer-readable storage medium or are held on a network or server, from where they can be loaded into the processor of the respective computing unit, which can be directly connected to the computing unit or can be embodied as a part of the computing unit. Furthermore control information of the computer program products can be stored on a computer-readable storage medium. The control information of the computer-readable storage medium can be embodied in such a way that, when the data medium is used in a computing unit, it carries out an inventive method. Examples of a computer-readable storage medium are a DVD, a magnetic tape or a USB stick, on which electronically readable control information, in particular software, is stored. When this control information is read from the data medium and stored in a computing unit, all forms of embodiment of the method described above can be carried out. In this way the invention can also be based on the the computer-readable medium and/or the the computer-readable storage medium. The advantages of the proposed computer program products or of the associated computer-readable media essentially correspond to the advantages of the proposed method.

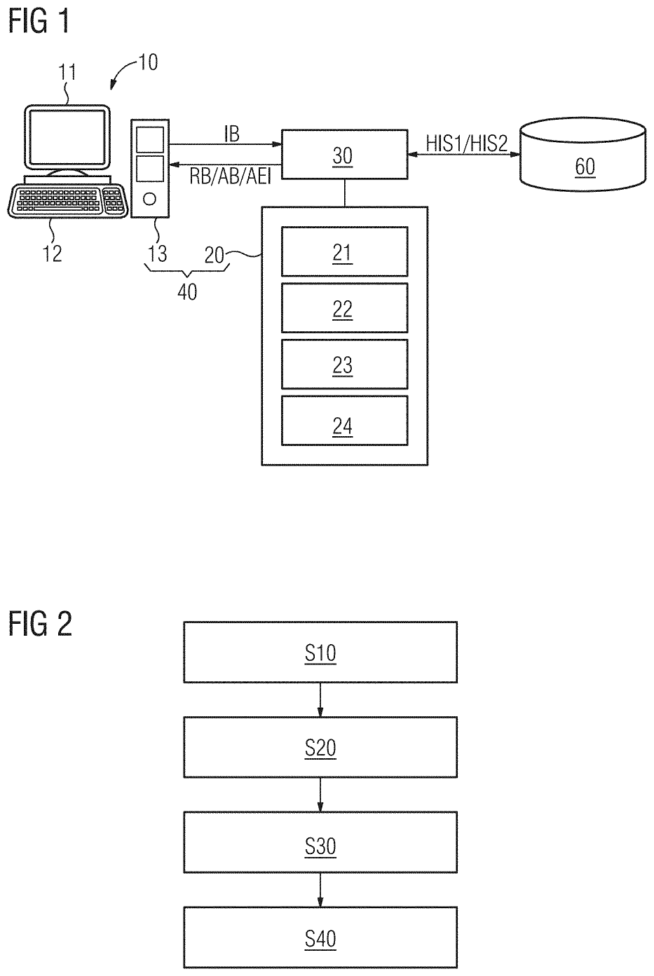

Shown in FIG. 1 is a system 1 for provision of similarity information AEI based on histopathology image data HIS1, HIS2 in accordance with a form of embodiment. The system 1 has a user interface 10, a computing unit 20, an interface 30, and a memory unit 60. The computing unit 20 is basically embodied for computation and provision of similarity information AEI based on histopathology image data HIS1, HIS2. The histopathology image data HIS1, HIS2 can be provided to the computing unit 20 via the interface 30 from the memory unit 60.

The memory unit 60 can be embodied as a central or local database. The memory unit 60 can in particular be part of a server system. The memory unit 60 can in particular be part of a medical information system such as a hospital information system (or HIS for short) and/or of a PACS system (PACS stands in this case for picture archiving and communication system) and/or of a laboratory information system (LIS).

Archived in the memory unit 60 is histopathology image data HIS1, HIS2. Histopathology image data HIS1, HIS2 is image data that is based on a tissue sample of a patient, which was taken from the latter at a certain point in time from an anatomical target or removal region. The anatomical removal region can for example be part of an organ or a tissue region that has been identified by an imaging modality such as an MR or CT device, for example. The tissue sample was taken from the patient for example in the course of a biopsy, an operation as operation preparate or excision. Micrometer-thick tissue slides are created from the tissue samples. Typically a number of regions (known as punch biopsies or blocks) are punched from a tissue sample with a punch cylinder, which are then cut into thin slices. The tissue slides arising can be fixed, prepared and readied by different techniques, before they are finally stained by histopathological staining. Histological stains serve on the one hand to increase the contrast of the tissue and cell structures contained in the slides. On the other hand histological stains can be explicitly employed to highlight specific features and thus to address specific pathological issues. There are a plurality of different histological stains, which have been developed in the course of the last 120 years. In first place there is mostly Hematoxylin-Eosin staining (H&E staining) as routine and overview staining. Special frequently mentioned histological stains going beyond this are e.g. Kongorot, Trichrome stains or Auramin O. Immunhistochemical stains can also be used as well, with which proteins or other structures can be made visible with the aid of marked antibodies. Examples of this are Ki67 as cell proliferation markers, Her2 immunostains as specific markers for breast cancer, CD8 immunostains for marking of T cells, or PD-L1 immunostains as predictive markers for the success of immunotherapies. In modern laboratories, at least for widely used stains, computer-assisted automatic staining systems are mostly employed. Mostly a first tissue slide of a block is stained with an H&E staining. If necessary and depending on the issue, in the follow-up to the appraisal, the tissue slides stained with H&E and further tissue slides of the respective blocks are stained with special stains and analyzed.

For appraisal the ready-prepared and stained tissue slides are frequently digitized nowadays. Specialized scanners, known as slide scanners, are used for this. The image recorded by them is also known as a whole slide image. The image data recorded by them is typically two-dimensional pixel data, wherein each pixel is assigned a color value.

Since a number of punch biopsies are typically taken from a tissue sample, which are treated with the same histopathological staining, the histopathology image data HIS1, HIS2 typically has a number of individual images (a number of individual whole slide images or a number of individual pixel images).

In addition histopathology image data HIS1, HIS2 can have metadata, in which individual information for the respective histopathology image data HIS1, HIS2 can be stored. For example the metadata can have one or more of the following items of information: a point in time at which the tissue sample the underlying the respective histopathology image data HIS1, HIS2 was taken from the patient, an electronic identifier identifying the patient, such as for example a patient ID or a name, a specification of which histopathological staining has been used for the respective histopathology image data HIS1, HIS2, a specification about an earlier appraisal of the histopathology image data HIS1, HIS2, an identifier identifying a user making the appraisal (e.g. the name or a user ID), a specification of a region indicating a pathological appraisal in the respective histopathology image data HIS1, HIS2, such as for example a region of interest IB, and/or a specification about the anatomical removal region of the patient, from which the tissue sample underlying the respective histopathology image data HIS1, HIS2 was taken. The metadata can be stored in a header of the histopathology image data HIS1, HIS2 or in a data container of the histopathology image data HIS1, HIS2 separate from the actual image data. As an alternative or in addition such metadata can also be stored in an Electronic Medical Record or EMR for short) of the patient, i.e. separately from the histopathology image data HIS1, HIS2. Such electronic medical records can be archived for example in the memory facility 60 or in a memory facility set up separately therefrom, to which the computing unit 20 can be connected via the interface 30.

The user interface 10 has a display unit 11 and an input unit 12. The user interface 10 can be embodied as a portable computer system, such as a smartphone, tablet computer, or laptop. The user interface 10 can further be embodied as a desktop PC. The input unit 12 can be integrated into the display unit 11, for example in the form of a touch-sensitive screen. As an alternative thereto or in addition, the input unit 12 can have a keyboard or a computer mouse or and/or a digital stylus. The display unit 11 is embodied to display single or multiple images from the histopathology image data HIS1, HIS2 (these displayed individual images are also referred to as reference images RB below), of similarity information AEI or assistance images AB established, with the assistance images AB illustrating to the user the similarity information AEI. The user interface 10 is further embodied to receive an input from the user in respect of the region of interest IB that is relevant for an appraisal. The user in this case can be a doctor and in particular a pathologist.

The user interface 10 has one or more processors 13, which are embodied to execute software for activating the display unit 11 and the input unit 12, in order to provide a graphical user interface, which makes it possible for the user to select histopathology image data HIS1, HIS2 for an appraisal, to enter regions of interest IB and to assess the similarity information AEI found. The user can activate the software via the user interface 10 for example, by downloading from an app store for example. In accordance with further forms of embodiment the software can also be a client-server computer program in the form of web application, which runs in a browser.

The interface 30 can have one or more individual data interfaces, which guarantee the exchange of data between the components 10, 20, 60 of the system 1. The one or more data interfaces can be part of the user interface 10, of the computing unit 20 and/or of the memory unit 60. The one or more data interfaces can have a hardware and/or software interface, e.g. a PCI bus, a USB interface, a Firewire interface, a ZigBee or a Bluetooth interface. The one or more data interfaces can have an interface of a communication network, wherein the communication network can have a Local Area Network (LAN), for example an Intranet or a Wide Area Network (WAN). Accordingly the one or more data interfaces can have a LAN interface or a Wireless LAN interface (WLAN or Wi-Fi).

The computing unit 20 can have a processor. The processor can have a Central Processing Unit (CPU), a Graphics Processing Unit (GPU), a Digital Signal Processor (DSP), an image processing processor, an integrated (digital or analog) circuit or combinations of the aforementioned components and further facilities for processing of histopathology image data HIS1, HIS2 in accordance with forms of embodiment of the invention. The computing unit 20 can be implemented as an individual component or have a number of components, which work in parallel or serially. As an alternative the computing unit 20 can have a real or virtual group of computers, such as for example a cluster or a cloud. Such a system can be called a server system. Depending on the form of embodiment the computing unit 20 can be embodied as a local server or as a cloud server. The computing unit 20 can further have main memory, such as a RAM, in order for example temporarily to store the histopathology image data HIS1, HIS2. As an alternative such main memory can also be embodied in the user interface 10. The computing unit 20 is embodied in such a way, e.g. through computer-readable instructions, through design and/or hardware, that it can execute one or more method steps in accordance with forms of embodiment of the present invention. In particular the computing unit 20 can be embodied to execute one or more image processing algorithms TF-A, TF-B, TF-C, TF-A′ described in greater detail below.

The computing unit 20 can have subunits or modules 21-24, which are embodied to provide a user, as part of an ongoing human-machine interaction, with similarity information AEI and in this way support to them in their appraisal.

The module 21 is embodied for provision of histopathology image data, from which new findings are to be obtained (depending on point in time of the tissue sample removal either the histopathology image data labeled HIS1 or HIS2). For example module 21 can be embodied to receive such histopathology image data HIS1 or HIS2 from the memory unit 60 and load it into the computing unit 20 or the user interface 10. This can occur for example in response to a user's command input via the user interface 10 or can be triggered automatically. The module 21 can further be embodied to display to the user in response to a command individual images of the histopathology image data HIS1 or HIS2 to be investigated as reference images RB via the user interface 10. The module 21 can further be embodied to establish a region of interest IB within the histopathology image data HIS1 or HIS2, with the region of interest IB being able to indicate a pathological appraisal. For this the module 21 can for example receive a corresponding user input from the user interface 10, evaluate an annotation by the user present in the histopathology image data HIS1 or HIS2, and/or determine the region of interest IB automatically. In respect of the last-mentioned alternative the module 21 can be embodied to apply a suitable image processing algorithm to the histopathology image data HIS1 or HIS2 (for example the second image processing algorithm TF-B described below).

The module 22 is embodied to provide histopathology image data, with which the histopathology image data to be appraised can be compared, in order to make a statement about the progression of a tumor illness of a patient (depending on the point in time of the tissue sample removal either the histopathology image data labeled HIS1 or HIS2). To this end the module 22 can be embodied in particular to search for histopathology image data HIS1 or HIS2 of the patient from past examinations, which preferably show tissue from the same tissue from the same or at least one similar anatomical removal region of the patient. The module 22 can be embodied to formulate a search query and to search through the memory facility 60 for example. Similarly to the module 21, module 22 can likewise be embodied to identify regions of interest IB in the comparison histopathology image data HIS1 or HIS2—for example by evaluating an annotation already present from an earlier appraisal or by applying a suitable image processing algorithm.

The module 23 is embodied, in the histopathology image data HIS1 or HIS2 to be examined and/or the histopathology image data HIS1 or HIS2 from earlier examinations, in particular to establish morphological and/or structural and/or texture-related similarities between the tumor tissue or tissue cells shown in each case. Such similarities can for example show whether a tumor visible in the new histopathology image data HIS1 or HIS2 to be examined is a recidive of a tumor already mapped in the old histopathology image data HIS1 or HIS2, or whether it involves a newly arisen tumor. The module 23 can be embodied to provide similarities recognized as similarity information AEI. In particular the module 23 can be embodied to apply suitable image processing algorithms to the histopathology image data HIS1, HIS2 in order to obtain similarity information AEI. For example the module 23 can be embodied to apply one or more of the image processing algorithms TF-A, TF-C or TF-A′ described below.

Module 24 can be construed as a visualization module, which is designed to display to the user the result of the similarity analysis from module 23 e.g. via the user interface 10. For this, module 24 can be embodied to provide the user with one or more assistance images AB based on the histopathology image data HIS1, HIS2, in which regions of similarity AEB are highlighted graphically and/or in color and/or in other ways. Regions of similarity AEB in this case, as mentioned, are those regions in the histopathology image data HIS1 or HIS2 that, starting from the previous tissue sample to be newly appraised, show a great similarity in the nature of the tumor tissue/tissue cells. In addition module 24 can be embodied to archive the results of the similarity analysis of module 23 (e.g. in the memory unit 60 or any other given memory unit) or provide them to a further module or further software for further processing.

The subdivision of the computing unit 20 into elements 21-24 undertaken serves in this case merely to explain in more simple terms how the computing unit 20 functions and is not to be understood as restrictive. The elements 21-24 or their functions can also be grouped together in one element. The elements 21-24 can in this case also in particular be construed as computer program products or computer program segments, which on execution in the computing unit 20 realize one or more of the method steps described above.

The computing unit 20 and the processor 13 can together form the controller 40. It should be noted that the layout of the controller 40 shown, i.e. the subdivision into the computing unit 20 and the processor 13, is likewise only to be understood by way of example. In this way the computing unit 20 can be integrated completely into the processor 13 and vice versa. In particular the method steps can run entirely on the processor 13 of the user interface 10 by executing a corresponding computer program product (e.g. software installed on the user interface), which then interacts via the interface 30 directly e.g. with the memory unit. In other words the computing unit 20 would then be identical to the processor 13.

As already mentioned the computing unit 20, in accordance with a few forms of embodiment, can alternatively be construed as a server system, such as e.g. a local server or a cloud server. With an embodiment of this type the user interface 10 can be referred to as “frontend” or “client”, while the computing unit 20 can then be construed as “backend”. Communication between the user interface 10 and the computing unit 20 can then be carried out for example based on an https protocol. The processing power in such systems can be divided between the client and the server. In a “thin client” system the server has the greater part of the processing power available to it, while the client in a “thick client” system provides more processing power. The same applies to the data (here: in particular the histopathology image data HIS1, HIS2). While in the “thin client” system the data remains mostly on the server and only the results are transferred to the client, data is also transferred to the client in the “thick client” system.

In accordance with further forms of embodiment the functionality described can also be provided by what is known as a cloud service. The correspondingly embodied computing unit is then embodied as a cloud platform. The data to be analyzed, i.e. the histopathology image data can then be uploaded to this cloud platform.

Shown in FIG. 2 is a schematic flow diagram of a method for provision of similarity information AEI based on histopathology image data HIS, HIS2 from tissue samples of a patient taken at different times. The sequence of the method steps is not restricted either by the order shown or by the numbering chosen. This means that the sequence of the steps can be changed where necessary and individual steps can be left out.

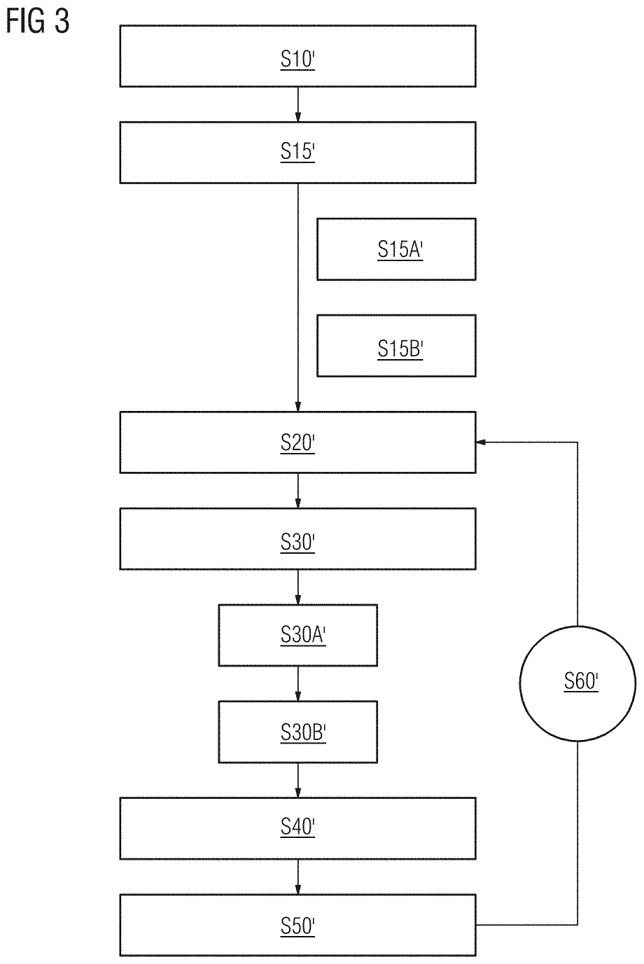

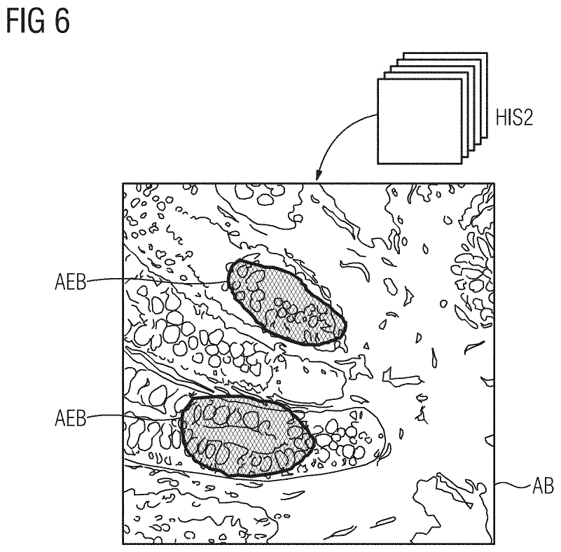

In FIG. 2 the similarity information AEI is created based on a comparison of first histopathology image data HIS1 and second histopathology image data HIS2. The first histopathology image data HIS1 in this case is created based on a tissue sample of the patient, which has been taken from the patient at a first point in time. The second histopathology image data HIS2 is based on a tissue sample of the same patient, which has been taken from the patient at a second point in time different from the first point in time. The tissue samples in this case have each be taken from the same or from at least one similar anatomical target region of the patient. FIG. 2 in this case represents the general case, which merely starts from different first and second points in time. In such cases the first histopathology image data HIS1 can be based on a tissue sample, which was taken from the patient before the tissue sample on which the second histopathology image data HIS2 is based—or vice versa. Further concrete examples of these two options will then be shown in FIGS. 3 and 6 shown below.

In the general case of FIG. 2, a first step S10 is directed to the provision of first histopathology image data HIS1. The provision in this case can be realized by retrieving the first histopathology image data HIS1 from the memory unit 60 and/or loading the first histopathology image data HIS1 into the computing unit 20.

A second step S20 is directed to the provision of second histopathology image data HIS2. The provision in this case can likewise be realized by retrieving the second histopathology image data HIS2 from the memory unit 60 and/or loading the second histopathology image data HIS2 into the computing unit 20.

In the next step S30 similarity information AEI is created. To this end the first histopathology image data HIS1 and the second histopathology image data HIS2 are input into a first image processing algorithm TF-A. This “matches” similar regions in the first histopathology image data HIS1 and the second histopathology image data HIS2 based on a defined similarity metric. The similarity metric in this case can in particular be a measure for morphological and structural similarities of the regions. The similar regions in this case can be in particular be regions indicating a pathological appraisal. For example such regions indicating a pathological appraisal can be those regions in the first histopathology image data HIS1 and second histopathology image data HIS2, which have tumor tissue or tumor cells or general pathological tissue changes. The fact that the similar regions establish a similarity relationship between tissue samples taken at different points in time enables it to be deduced whether the regions in the first histopathology image data HIS1 and the second histopathology image data HIS2 indicating a pathological appraisal are related to one another. If for example regions have been identified in the first histopathology image data HIS1, which are similar to one or more regions in the second histopathology image data HIS2, morphologically similar pathological tissue changes are present. This in its turn indicates that pathological tissue changes in the first histopathology image data HIS1 represent a recidive of pathological tissue changes in the second histopathology image data HIS2 or vice versa (depending on which tissue sample underlying the respective histopathology image data HIS1 or HIS2 was taken earlier).

Accordingly the similarity information AEI can contain information about whether a recidive relationship exists between the first histopathology image data HIS1 and the second histopathology image data HIS2. The similarity information AEI can further contain location information about the similar regions in the first histopathology image data HIS1 and/or the second histopathology image data HIS2 indicating a pathological appraisal, with the location information for example showing a surrounding box and/or coordinates of the similar region. The similarity information AEI can further contain a specification in respect of the degree of similarity between the similar regions of the first histopathology image data HIS1 and the second histopathology image data HIS2. The similarity information AEI can further contain one or more assistance images AB, which can be based on the first histopathology image data HIS1 and/or the second histopathology image data HIS2 and in which e.g. similar regions indicating a pathological appraisal can be highlighted.

In a further step S40 the similarity information AEI is finally provided. Provided can mean in general that the similarity information AEI is made available for use. For example the similarity information AEI can be displayed to the user via the user interface 10. In addition or as an alternative the similarity information AEI can be archived in the memory unit 60 or input into a further algorithm for further processing.

In an optional step S50 a medical report or appraisal report is finally created automatically based upon the similarity information AEI. This can comprise a suitable template being pre-filled with the similarity information AEI and provided to the user for their attention and further processing via the user interface 10.

For further illustration, in the form of embodiment shown in FIG. 3, the case is now discussed in which the first histopathology image data HIS1 is based on a tissue sample taken later than the second histopathology image data HIS2. In other words the second point in time this case lies chronologically before the first point in time and the first histopathology image data HIS1 can be viewed as a follow-up to the second histopathology image data HIS2. The sequence of the method steps is not restricted either by the order shown or by the numbering chosen. This means that the sequence of the steps can be changed where necessary and individual steps can be left out.

A first step S10′ is directed to the provision of the first histopathology image data HIS1. The first histopathology image data HIS1 can for example be selected by a user in the user interface 10. The computing unit 20 can then retrieve the first histopathology image data HIS1 from the memory unit 60 and load it into a main memory or into another memory facility of the computing unit 20.

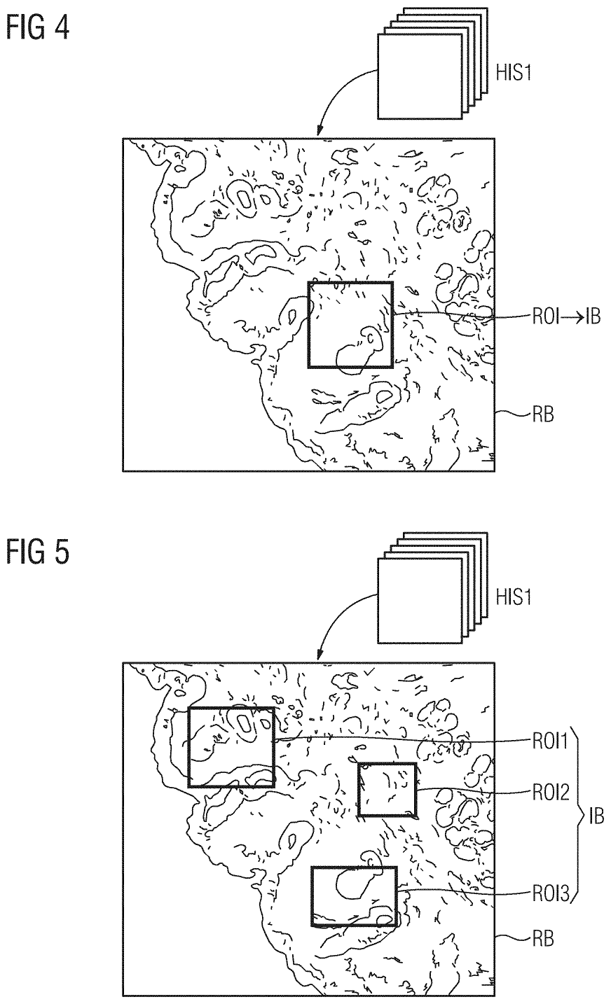

Then, in a next step S15′, a region of interest IB in the first histopathology image data HIS1 is determined. The region of interest IB is in particular a region in (or an extract from) the first histopathology image data HIS1, which shows a morphology relevant for a pathological appraisal. In other words the region of interest can be construed as a region (or extract) of the first histopathology image data HIS1 indicating a pathological appraisal. The region of interest IB in this case can have one or more individual regions (or extracts) ROI, ROI1, ROI2, etc. (cf. FIGS. 4 and 5). In accordance with a few implementations the region of interest IB can furthermore also comprise the entire first histopathology image data HIS1. The region of interest IB can be determined automatically (step S15A), or manually (step S15B).

For automatic determination of the region of interest IB in step S15A, the first histopathology image data HIS1 can be input into a second image processing algorithm TF-B, which is embodied to identify in histopathology image data HIS1, HIS2 regions relevant for the pathological appraisal, i.e. in particular regions showing pathological tissue changes. As explained further below, the second image processing algorithm TF-B can in particular have a trained function.

For manual determination of the region of interest IB in step S15B, the system 1 can be embodied in such a way that the user can select from the first histopathology image data HIS1 one or more reference images RB and can assess them in the user interface 10. The system 1 and in particular the user interface 10 can further be embodied in such a way that the user can identify the region of interest IB with an annotation, with the user being able to enter the annotation with a user input via the user interface 10. For example the system 1 can be embodied in such a way that the user can annotate the region of interest IB by marking regions relevant for them in one or more reference images RB with a mouse click or by drawing a frame around them.

As well as this, a semi-automatic determination of the region of interest IB is also conceivable, in which possibly relevant regions are determined automatically by the second image processing algorithm TF-B and displayed to the user via the user interface 10 for selection. The regions confirmed by the user will then be taken over for the region of interest IB.

A next step S20′ is then directed to the provision of the second histopathology image data HIS2, which is based on tissue samples that were taken from the patient before the tissue sample removal for the first histopathology image data HIS1. To this end the computing unit 20 can be embodied in such a way that it searches the memory unit 60 for suitable second histopathology image data HIS2 and loads the image data. In accordance with a few forms of embodiment the second histopathology image data HIS2 found in this way can be displayed to the user for selection. In this way the user is put in a position to select from a preselection second histopathology image data HIS2 that they themselves see as well suited. In accordance with forms of embodiment of the invention the second histopathology image data HIS2 in question is presented to the user in the form of a timeline in a graphical user interface for selection, which gives the user a rapid and comprehensive overview. Individual points on the timeline in this case can specify the second histopathology image data HIS2 available. As an alternative the computing unit 20 can be embodied to select suitable second histopathology image data HIS2 autonomously. As an alternative the system 1 and in particular the user interface 10 can also be embodied in such a way that the user selects the second histopathology image data HIS2 entirely by themselves, such as by independently searching through the histopathology image data stored in the memory unit 60.

In order to be suitable for establishing the similarity information AEI in accordance with forms of embodiment of the invention, the second histopathology image data HIS2 should at least be assigned to the same patient as the first histopathology image data HIS1. The probability of finding similar regions to the region of interest IB and thus providing meaningful similarity information AEI, increases moreover if the second histopathology image data HIS2 is further based on a tissue sample that has been taken from the same or at least one similar anatomical target region to the tissue sample from which the first histopathology image data HIS1 was created. Moreover better results can be achieved if the same staining has been used for the first histopathology image data HIS1 and the second histopathology image data HIS2. In addition a suitable period of time between first and second points in time can be of relevance. This information can be taken into account in the provision of the second histopathology image data HIS2 as metadata. This metadata can be extracted automatically for example from the first histopathology image data HIS1, can be retrieved automatically from a medical information system and/or can be provided by the user. For example information relating to the patient, the anatomical target region or the staining can be stored in a header of the first histopathology image data HIS1 and extracted from there. As an alternative this information can be obtained by retrieving it from electronic patient records stored in a medical information system. In addition the user can be asked for the metadata, such as by providing a corresponding input mask via the user interface 10. As well as this a suitable time window for the periods of time between the first and the second point in time can be predetermined.

In accordance with a few forms of embodiment there can also be provision for taking account of the region of interest IB determined in step S15 and/or of information derived therefrom (such as for example a feature signature—see below) in the provision of the second histopathology image data HIS2. This can be sensible in particular when such regions of interest IB from previous findings/analyses are also already annotated in the second histopathology image data HIS2.

The next step S30′ is directed to the establishment of the similarity information AEI. To this end there is provision, in a substep S30A′, for searching through the second histopathology image data HIS2 for regions of similarity AEB. To this end the second histopathology image data HIS2 can be input into a third image processing algorithm TF-C. Optionally the first histopathology image data HIS1 and/or the regions of interest IB can also be input into the third image processing algorithm TF-C. The third image processing algorithm TF-C can generally be embodied to search in histopathology image data for regions that exhibit a similarity with predeterminable image data. In the present case this predeterminable image data is given by the region of interest IB. To establish whether a similarity exists between image data, the third image processing algorithm TF-C can further be embodied to apply a defined similarity metric, as will be explained further below.

In other words the regions of similarity AEB are the regions of the second histopathology image data HIS2 indicating a pathological appraisal. The regions of similarity AEB can have a morphological similarity to the region of interest IB. The regions of similarity AEB can further have similar patterns or structures to the region of interest IB. The regions of similarity AEB can further have a similar feature signature to the region of interest IB. A feature signature can be understood for example as a set or vector of abstract features, which can be extracted from the image and/or metadata. For the search for regions of similarity the second histopathology image data HIS2 can be ‘scanned’ for example and the image data of the second histopathology image data HIS2 can in this way be compared step-by-step with the image data of the region of interest IB. The regions of similarity AEB can in particular have a similarity to the region of interest IB that lies above a predetermined similarity level or measure of similarity. The similarity level or measure of similarity can specify a degree of similarity between different image data. The similarity that is compared with the predetermined measure of similarity can be the result of the above-mentioned similarity metric. The predetermined measure of similarity can be predetermined manually or automatically for example. It is to be noted that the search for regions of similarity AEB can also produce a negative result if no regions that are similar to the region of interest IB are present in the second histopathology image data HIS2.

Optionally, in step S30A′, further account can be taken of existing annotations in the second histopathology image data HIS2, which already indicate a pathological appraisal from an earlier appraisal (i.e. thus “regions of interest” in the second histopathology image data HIS2).

Based on the first substep S30A′, in a second substep S30B′ the similarity information AEI is then determined. The similarity information AEI can for example have a specification about the regions of similarity AEB, such as for example their location in the second histopathology image data HIS2, their size, a quantitative specification about their similarities etc. If in step S30B′ no regions of similarity AEB were found, this can be specified accordingly in the similarity information AEI. Furthermore the similarity information AEI can comprise a visualization for the user. A visualization in this case can be based on the second histopathology image data HIS2 for example. In particular an assistance image AB can be created based on the second histopathology image data HIS2, in which one or more regions of similarity AEB are highlighted (cf. FIG. 6). A highlight in this case can for example be provided by a frame and/or by a colored contrasting marking of the regions of similarity. In respect of the colored contrasting marking it is furthermore possible to show the similarity of the individual regions of similarity AEB by a color code, in which graduations in the similarity values calculated for the individual regions of similarity AEB are assigned to a color gradient. As an alternative or in addition the similarity information AEI can have information about a probability that the region of interest IB is in a recidive relationship with the regions of similarity AEB. This can show that the pathological tissue changes shown in the region of interest IB are a recidive of the pathological tissue changes shown in the regions of similarity AEB, which can point to a resurgence of a disease. As well as regions of similarity AEB, the second histopathology image data HIS2 can have still further regions indicating a pathological appraisal, which although they show a pathological tissue change for example, have no similarities with the regions of the first histopathology image data HIS1 indicating a pathological appraisal. These regions can likewise be highlighted in the assistance image AB and preferably highlighted differently from the regions of similarity AEB—such as by a highlight in another color.

In a step S40′ the similarity information AEI is finally provided. Step S40′ in this case essentially corresponds to step S40 and the actions described in step S40 can also be undertaken in step S40′. In particular the assistance image AB can be displayed to the user in step S40′ via the user interface 10. As an alternative or in addition the assistance image AB can be archived in the memory unit 60.

The optional step S50′ essentially corresponds to step S50 from FIG. 2. In particular, in step S50′, the assistance image AB and/or metadata from the first histopathology image data HIS1 and/or second histopathology image data HIS2 and/or information about a recidive probability can be entered automatically into a template of a medical appraisal report or a report, which is then made available to a user via the user interface 10 or can be archived in the memory unit 60.

The optional step S60′ is a repetition step. Step S60′ takes account of the fact that, depending on the medical history of the patient, a number of items of second histopathology image data HIS2 can be considered for a reconciliation with the “current” first histopathology image data HIS1. As explained in conjunction with step S20′, second histopathology image data HIS2 coming into consideration can in fact be presented for selection. Depending on implementation however there is no absolute provision for this. Moreover the user can possibly select a number of items of second histopathology image data HIS2. In these cases a number of items of second histopathology image data HIS2 are present, which come into consideration for the analysis of the subsequent steps. Therefore there can be provision in the optional step S60′ for the steps S20′, S30′, S40′ and S50′ to be repeated for the different second histopathology image data HIS2, wherein other second histopathology image data HIS2 is the basis for each pass until all second histopathology image data HIS2 coming into consideration has been completely processed.

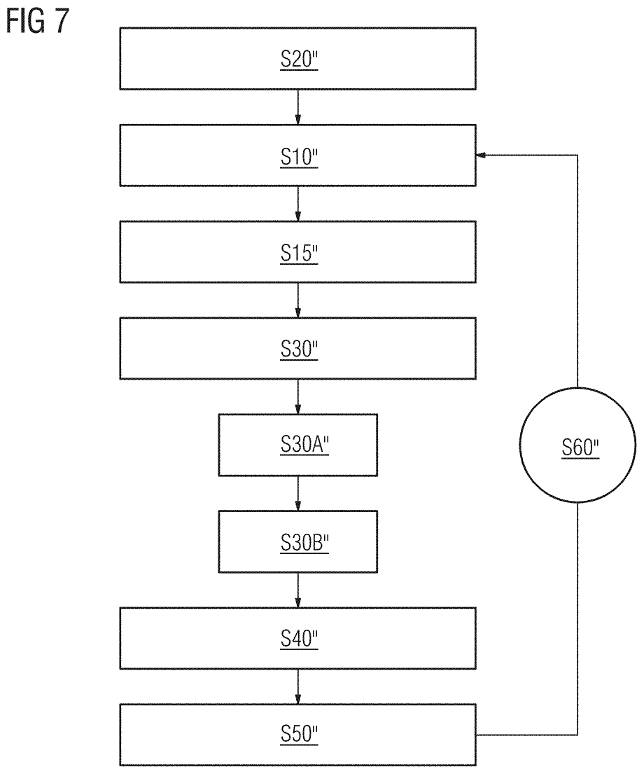

Shown in FIG. 7 is a further form of embodiment of a method for provision of similarity information AEI for histopathology image data HIS1, HIS2. The sequence of the method steps is not restricted either by the order shown or by the numbering selected. This means that the sequence of the steps can be changed if necessary and individual steps can be left out. Unlike the form of embodiment shown in FIG. 3, in this form of embodiment the first point in time lies before the second point in time. In other words the first histopathology image data HIS1 refers back to a tissue sample, which was taken from the patient chronologically before the tissue sample to which the second histopathology image data HIS2 refers back. The second histopathology image data HIS2 is thus this time a result of a follow-up examination just available for appraisal, while the first histopathology image data HIS1 belongs to prior examinations (known as “priors”). As a further difference, in the form of embodiment shown in conjunction with FIG. 7, it is assumed that in the first histopathology image data HIS1 relevant regions are already defined by earlier findings that indicate a pathological appraisal and can represent the basis for the regions of interest IB.

In a first step S20″ the second histopathology image data HIS2 is first of all provided. Step S20″ essentially corresponds, in respect of its technical implementation, to step S10′ from FIG. 3. Accordingly the individual steps, alternatives, explanations and effects described in conjunction with step S10′ are able to be applied similarly to step S20″.

Then, in step S10″, with the first histopathology image data HIS1, suitable reference data from earlier examinations of the patient is sought. Step S10″ in this case technically essentially corresponds to step S20′ from FIG. 3. The individual steps, alternatives, explanations and effects described in conjunction with the provision of the second histopathology image data HIS2 in step S20′ are able to be applied similarly to the provision of the first histopathology image data HIS1 according to S10″.

Unlike in FIG. 3, in step S15″ the region of interest IB is not defined in the “follow-up” histopathology image data but in the “priors”. This is the form of embodiment of the first histopathology image data HIS1 shown in FIG. 7. The region of interest IB in this case is again a region that shows a pattern relevant for a pathological appraisal. In particular the region of interest IB in this case can again have one or more individual regions ROI, ROI1, ROI2, etc. (cf. FIGS. 4 and 5). The region of interest IB is preferably determined automatically in step S15″. For automatic determination of the region of interest IB the first histopathology image data HIS1 can be input into the already mentioned second image processing algorithm TF-B, which is embodied to identify in histopathology image data HIS1, HIS2 regions relevant for the pathological appraisal, i.e. in particular pathological tissue changes such as regions showing tumor tissue. Alternatively already existing annotations can be evaluated in the first histopathology image data HIS1, which for example a user has set up in the course of an earlier appraisal in the first histopathology image data HIS1. These annotations can be stored for example as markings of regions of interest IB in the first histopathology image data HIS1 or as associated metadata.

The next step S30″ is directed to the establishment of the similarity information AEI. To this end there is provision in a substep S30A″ for searching the second histopathology image data HIS2 for regions of similarity AEB. As a systematic difference from the form of embodiment shown in FIG. 3, the regions of similarity AEB are thus not sought in the “priors” HIS1, but in the “follow-up” data HIS2. In respect of its technical implementation, step S30A″ in this case essentially corresponds to step S30A′ from FIG. 3 and the individual steps, alternatives, explanations and effects described in conjunction with step S30A′ are able to be transferred similarly to step S30A″.

Based on the substep S30A″, in a second substep S30B″ the similarity information AEI is then determined. Step S30B″ in this case essentially corresponds to step S30B′ from FIG. 3 and the individual steps, alternatives, explanations and effects described in conjunction with step S30B′ are able to be transferred similarly to step S30B″. In particular, the form of embodiment shown in FIG. 7 can likewise be provided to create an assistance image AB, which is based on the second histopathology image data HIS2. As a systematic difference, in the form of embodiment shown in FIG. 7, an assistance image AI would however preferably be created based upon the follow-up examination.

The steps S40″ and S50″ essentially correspond to the steps S40′ and S50′ from FIG. 3 and the individual steps, alternatives, explanations and effects described in conjunction with the steps S40′ and S50′ are able to be transferred similarly to step S40″ and S50″.

The optional step S60″ is finally a repetition step similar to step S60′—with the difference that step S60″ is directed to the first histopathology image data HIS1. Accordingly there is provision in the optional step S60″ for the steps S10″, S15″, S30″, S40″ and S50″ to be repeated for different first histopathology image data HIS1, wherein other first histopathology image data HIS1 is the basis for each pass until all first histopathology image data HIS1 coming into consideration has been completely processed.

It is pointed out that the two forms of embodiment shown in FIG. 3 and FIG. 7 are able to be combined with one another. Accordingly regions of interest IB can be defined both in the first histopathology image data HIS1 and also in the second histopathology image data HIS2, which can then be examined for similarities to determine regions of similarity AEB. Conversely the dedicated establishment of regions of interest IB can be dispensed with entirely and the first histopathology image data HIS1 and the second histopathology image data HIS2 can be analyzed as such and in their entirety with the aid of a suitable image processing algorithm TF-A for regions indicating a pathological appraisal, which are similar over the period between first and second point in time.

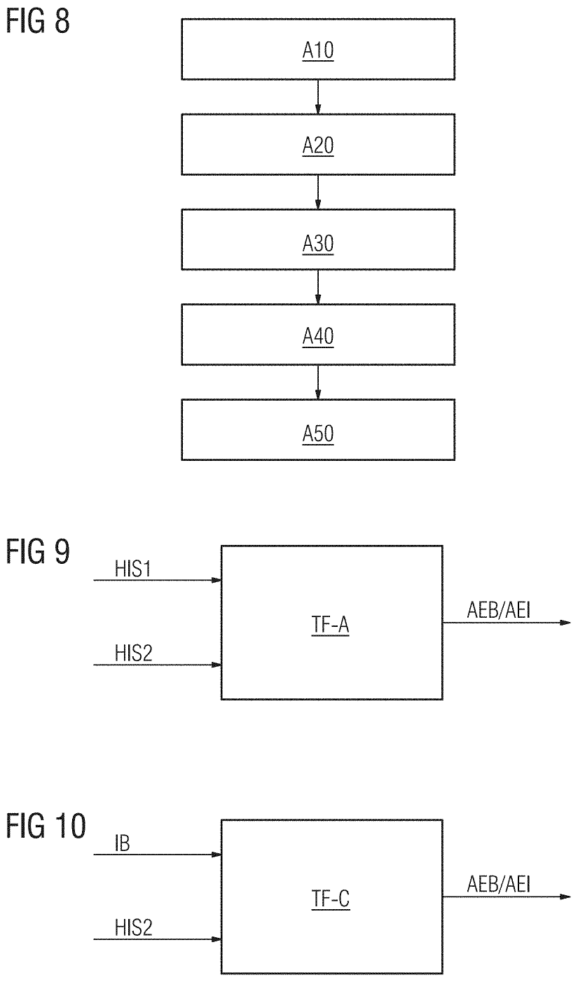

Shown in FIG. 8 is a method for determining regions of similarity in histopathology image data HIS1, HIS2. The sequence of the method steps is not restricted either by the order shown or by the selected numbering. This means that the sequence of the steps can be exchanged where necessary and individual steps can be left out. In particular the third image processing algorithm TF-C can be embodied to implement one or more of the steps explained in conjunction with FIG. 8.

The form of embodiment shown in FIG. 8 assumes that a region of interest IB is present. Thus the steps shown in FIG. 8 can for example follow on from the steps S15′ or S15″. Using this as its starting point, a first step A10 is directed to an extraction of a feature signature fIB based on the region of interest IB. The feature signature fIB can have a number of individual features, which were extracted from the region of interest IB and characterize the region of interest IB overall. The feature signature fIB can have a so-called feature vector, in which individual features are grouped together. If the region of interest IB is composed of a number of individual regions ROI, ROI1, ROI2, ROI3, the individual features of the feature signature fIB can be averaged over the individual regions. The features can for example comprise a pattern, a texture and/or a structure in the region of interest IB. Furthermore the features of the feature signature fIB can have parameters, which characterize the (cell) density and or the density of a histopathological marker in the region of interest IB. One or more features of the feature signature fIB can further have parameters that designate a color value, a grey scale or a contrast value in the region of interest IB. In addition one or more features of the feature signature fIB can be directed to characteristics that lie outside the region of interest IB. For example these can be information about surrounding tissue or information, which was taken from the metadata for the first histopathology image data HIS1. The feature signature fIB can be generated with a separate image processing algorithm, into which the region of interest IB and also optionally the first histopathology image data HIS1 and any metadata are input. For example what are known as texture classification algorithms (cf. e.g.: Hamilton et al., “Fast automated cell phenotype image classification,” BMC Bioinformatics, 8:110, 2007, DOI: 10.1186/1471-2105-8-110, the entire contents of which are hereby incorporated herein by reference) or trained functions, such as for example a convolutional neural network (see below), can be used for this. The aforementioned image processing algorithm can be implemented in particular as a subroutine of the third image processing algorithm TF-C.

In a next step A20 possible regions of similarity AEB are identified in the second histopathology image data HIS2. This can be done for example by systematic scanning of the second histopathology image data HIS2. In this case for example a “moving window” can be moved over the second histopathology image data HIS2 or the second histopathology image data HIS2 can be subdivided by a grid into possible regions of similarity AEB. As a further alternative possible regions of similarity AEB can also be defined dynamically, i.e. with variable size. Here contiguous regions can be identified based upon image values consistent in a region, such as grey scales, contrast, density, etc. As well as this an identification of possible regions of similarity is possible through an evaluation of image edges (cf. Zitnick et al., “Edge Boxes: Locating Object Proposals from Edges,” Computer Vision—ECCV, 2014, pp 391-405, the entire contents of which are hereby incorporated herein by reference). Moreover the use of a trained function and in particular a convolutional neural network is possible. As a further possibility a prioritization can already be undertaken in the identification of possible regions of similarity AEB and only those regions of the second histopathology image data HIS2 that come into consideration with a degree of probability as regions of similarity AEB can be identified as possible regions of similarity AEB. For this for example a segmentation can be used, which excludes less relevant regions, such as for example necrotic tissue regions, of the second histopathology image data HIS2 from the further analysis. As an alternative thereto or in addition, an algorithm similar to the second image processing algorithm TF-B can be applied, which is embodied automatically to recognize relevant regions in histopathology image data.

In a next step A30 the possible regions of similarity AEB of the feature signatures fAEB corresponding to feature signature fIB are extracted. The process in this case can essentially be as described in step A10.

In a next step A40 the extracted feature signatures fAEB of possible regions of similarity AEB are compared with the feature signature fIB of the region of interest IB. In this case a similarity metric can be determined in particular for each possible region of similarity AEB, with the similarity metric representing a measure for a similarity or a match between the feature signature fAEB extracted from the respective possible region of similarity AEB and the feature signature fIB of the region of interest IB. For example the similarity metric can be defined by a distance between the feature signatures in the feature space. If the feature signatures are construed as feature vectors, the similarity metric can be defined for example as a cosine similarity.

In step A50 the regions of similarity AEB are selected based on the similarity from the possible regions of similarity AEB. In this case for example all those possible regions of similarity AEB can be classified as regions of similarity AEB, of which the associated similarity metric has a measure of similarity with the region of interest IB, which lies above a defined threshold. The threshold can be defined automatically or manually.

Then for example the steps S30B′ or S30B″ can follow on from step A50.

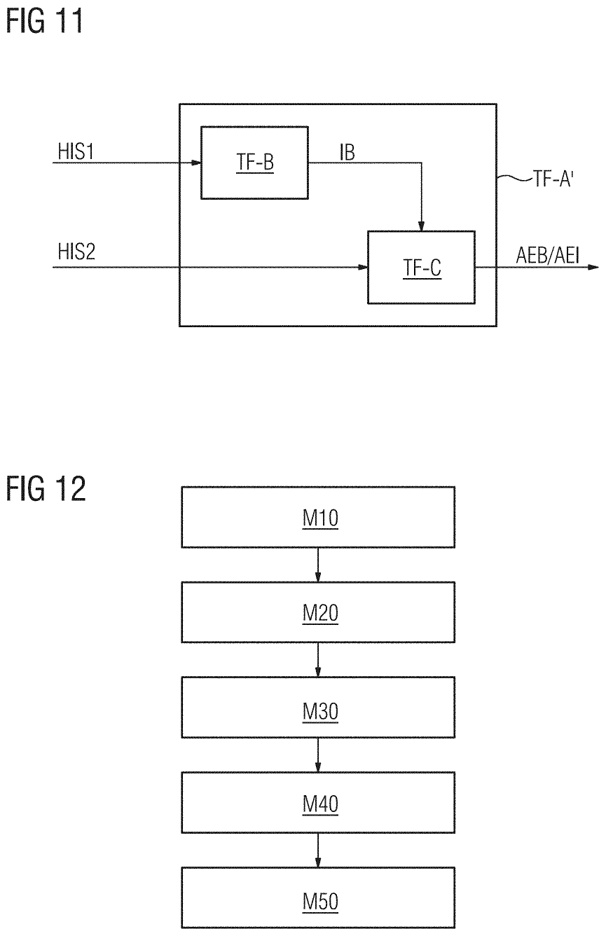

In accordance with forms of embodiment of the invention method steps of the forms of embodiment shown in FIG. 2, FIG. 3, FIG. 7 and FIG. 8 are executed by one or more image processing algorithms TF-A, TF-B, TF-C. FIGS. 9, 10 and 11 show forms of embodiment of these image processing algorithms. The image processing algorithm shown in FIG. 9 corresponds to the first image processing algorithm TF-A introduced in conjunction with FIG. 2. It receives the first histopathology image data HIS1 and the second histopathology image data HIS2 as input data and outputs as output data the regions of similarity AEB and/or the similarity information AEI. The image processing algorithm shown in FIG. 10 corresponds to the third image processing algorithm TF-C mentioned in conjunction with FIGS. 3 and 7. This receives the regions of interest IB and also the second histopathology image data HIS2 as input data and outputs the regions of similarity AEB and/or the similarity information AEI as output data. Shown in FIG. 11 is a variation TF-A′ of the first image processing algorithm TF-A. The image processing algorithm TF-A′ is characterized in that in it the second image processing algorithm TF-B and the third image processing algorithm TF-C are implemented as subroutines. As mentioned, the second image processing algorithm TF-B is embodied in such a way that that it recognizes the regions of interest IB in histopathology image data HIS1, HIS2. Optionally at least one part of the second image processing algorithm TF-B can be implemented in the third image processing algorithm TF-C.

In accordance with a few forms of embodiment the image processing algorithms TF-A, TF-B, TF-C, TF-A′ have one or more trained functions. These trained functions can have a neural network in accordance with forms of embodiment. Neural networks can have a plurality of consecutive layers. Each layer comprises at least one, preferably a number of nodes. Essentially each node can carry out a mathematical operation, which assigns an output value to one or more input values. The nodes of each layer can be connected to all or to just a subset of nodes of a previous and/or subsequent layer. Two nodes are “connected” when their inputs or outputs are connected. The edges or connections are associated with a parameter, which is frequently referred to as “weight” or “edge weight”. Input values for the nodes of the first layer in each case can for example be the pixel values of the first histopathology image data HIS1, or of the second histopathology image data HIS2 or of the regions of interest IB. The last layer in each case is often referred to as an output layer. Output values of the output layer, depending on image processing algorithm, can for example be pixel values or coordinates of the region of interest IB or of the regions of similarity AEB. Moreover the output values of the output layer can be similarity information AEI. Located between the input layer and the output layer are a number of hidden layers.

In accordance with a few forms of embodiment the trained functions can in particular have a convolutional neural network, abbreviated to CNN) or a deep convolutional neural network. Such trained functions then have one or more convolutional layers and, optionally, one or more deconvolutional layers. The trained function can further have pooling layers and upsampling layers as well as fully connected layers. Convolutional layers fold the input and forward its results to the next level, by an image filter being moved over the input. Convolutional layers can in particular prove to be advantageous when, as in a few forms of embodiment, similar image regions are to be searched for. The pooling layers reduce the dimensions of the data, by the outputs of groups of nodes of a layer being combined at an individual node in the next layer. Upsampling layers and deconvolutional layers reverse the actions of the convolutional layers and the pooling layers. Fully connected layers connect each node of preceding layers with nodes of subsequent layers, so that essentially each layer receives a “voice”.

In accordance with a few forms of embodiment the trained functions have what are known as region-based convolutional neural networks, or R-CNN for short). A difficulty in the search for regions of interest IB or regions of similarity AEB can be that such regions occur at different points in the histopathology image data HIS1 and HIS2 and can have different sizes and shapes. Although these problems can basically be addressed by the systematic “scanning” of the histopathology image data HIS1, HIS2 described in conjunction with step A20, this can often only be done with significant processing outlay and thus time. Essentially, region-based convolutional neural networks select at least a few selection regions from the image data to be analyzed (wherein here the techniques described in conjunction with step A20 can be applied). Then a convolutional neural network is used to extract feature signatures from the selection regions, based upon which the selection regions can be classified with a classifier. In such cases what are known as Support Vector Machines (or SVM for short) can frequently be used as classifiers or further neural network layers. For more comprehensive information about region-based convolutional neural networks the reader is referred by way of example to Girshick et al., “Rich feature hierarchies for accurate object detection and semantic segmentation,” arXiv:1311.2524, the entire contents of which are hereby incorporated herein by reference.

Based on this basic configuration a few further developments exist, which can likewise be implemented in the trained functions in accordance with forms of embodiment of the invention and for uniformity can likewise be referred to as region-based convolutional neural networks. When reference is made to one or more trained functions having a region-based convolutional neural network, this also includes the further developments described below and other further developments. One of these further developments is referred to as a fast region-based convolutional neural network (or fast R-CNN for short). Here feature signatures released from the selection regions are established for the entire image and then “pooled” depending on selection region. This enables multiple computations of feature signatures with overlapping selection regions to be eliminated (cf. Girshick, “Fast R-CNN,” 2015 IEEE International Conference on Computer Vision (ICCV), DOI: 10.1109/ICCV.2015.169, the entire contents of which are hereby incorporated herein by reference). A further development is referred to as a “faster” region-based convolutional neural network (or faster R-CNN for short). Here the selective choice of the selection regions is replaced by a selection via a (convolutional) neural network (cf. Ren et al., “Faster R-CNN: Towards Real-Time Object Detection with Region Proposal Networks,” Advances in Neural Information Processing Systems. Vol. 28, 2015, the entire contents of which are hereby incorporated herein by reference).

In accordance with a few forms of embodiment region-based convolutional neural networks can be implemented in particular for an automatic establishment of the regions of interest IB or for searching for regions of similarity AEB—i.e. thus in the second image processing algorithm TF-B and the third image processing algorithm TF-C wherein, in the search for regions of similarity AEB, classification is against the regions of interest IB or their feature signatures fIB. Of course the first image processing algorithm TF-A can also contain a region-based convolutional neural network.

As an alternative to region-based convolutional neural networks “conventional” convolutional neural networks can also be trained to have the same functional scope as region-based convolutional neural networks (i.e. to provide essentially the same output data). Such solutions are also referred to as YOLO (you only look once) solutions (cf. Redmon et al., “You Only Look Once: Unified, Real-Time Object Detection,” arXiv:1506.02640, the entire contents of which are hereby incorporated herein by reference).

A trained function learns by adaptation of weights or weighting parameters (e.g. of the edge weights) of individual layers and nodes. A trained function can be trained for example by supervised learning methods. Here for example the method of back propagation is used. During the training the trained function is applied to training input data in order to create corresponding output values, of which the target values are known in the form of training output data. The difference between the output values and the training output data can be used to introduce a cost or loss functional as a measure for how well or badly the trained function fulfills the task set for it. The aim of the training is to find a (local) minimum of the cost functional, by the parameters (e.g. the edge weights) of the trained function being iteratively adapted. The trained function is thereby ultimately put into a position to deliver acceptable results over a (sufficiently) large cohort of training input data. This optimization problem can be carried out using a stochastic gradient descent or other approaches known from the technical field.

For the first image processing algorithm TF-A shown in FIG. 9, if this has a trained function, training datasets would have first training histopathology image data HIS1 and second training histopathology image data HIS2 in each case, as well as, depending on the configuration of the first image processing algorithm TF-A, associated verified regions of similarity AEB or verified similarity information AEI. The first training histopathology image data HIS1 and the second training histopathology image data HIS2 in this case correspond to the first histopathology image data HIS1 or the second histopathology image data HIS2. In particular they thus belong to the same patient and are based on tissue samples that have been taken from the patient at different points in time but from the same anatomical target region. The verified regions of similarity AEB or similarity information AEI could in this case be based on an annotation of a user, who has made this based on an analysis appraisal of the first training histopathology image data HIS1 and the second training histopathology image data HIS2. In accordance with the nomenclature used here the first histopathology image data HIS1 and the second histopathology image data HIS2 would be the training input data and the target values or the training output data would be the verified similarity region AEB or similarity information AEI. A training of the first image processing algorithm TF-A could then comprise an application of the image processing algorithm TF-A to the first training histopathology image data HIS1 and second training histopathology image data HIS2 for creation of output values as well as a comparison of the output values with the verified regions of similarity AEB or similarity information AEI. Then, based on the comparison, one or more parameters of the first image processing algorithm TF-A can then be adapted.

For the second image processing algorithm TF-B suitable training datasets comprise training histopathology image data HIS1, HIS2 as well as verified regions of interest IB. Since it is a task of the second image processing algorithm TF-B automatically to recognize regions relevant for a pathological appraisal (i.e. regions indicating a pathological appraisal), the verified regions of interest IB can in particular be preserved by an annotation of a user, which for example designates tumor cells in the first histopathology image data HIS1. A training of the second image processing algorithm TF-B can then comprise an application of the second image processing algorithm TF-B to the training histopathology image data HIS1, HIS2 for creation of output values as well as a comparison of the output values with the verified regions of interest. Then, based on the comparison, one or more parameters of the second image processing algorithm TF-B can be adapted.

For the third image processing algorithm TF-C shown in FIG. 10 suitable training datasets accordingly each comprise training regions of interest IB and second training histopathology image data HIS2 as well as, depending on configuration of the third image processing algorithm TF-C, associated verified regions of similarity AEB or verified similarity information AEI. The training regions of interest can in this case in principle be any given regions extracted from histopathology image data. In particular in this case any given region of interest IB from the second histopathology image data HIS2 can be involved. In order to prepare the third image processing algorithm TF-C even better for the situation in the field, it is preferred that the training regions of interest IB indicate a pathological appraisal. Such training regions of interest IB can be annotated by a user for example. It is furthermore preferred for the training regions of interest IB to have been taken from histopathology image data, which, like the first histopathology image data HIS1, belongs to the same patient (and anatomical target region) as the second training histopathology image data HIS2, but are based on a tissue sample taken at a different point in time. For the verified regions of similarity AEB and verified similarity information AEI the process can be as described above. A training of the third image processing algorithm TF-C could then comprise an application of the third image processing algorithm TF-C to the training region of interest IB and the second training histopathology image data HIS2 for creation of output values as well as a comparison of the output values with the verified regions of similarity AEB or similarity information AEI. Then, based on the comparison, one or more parameters of the third image processing algorithm TF-C can be adapted.

Based on the third image processing algorithm TF-C a variation can further be created, which is in a position to recognize regions of similarity AEB within a histopathology image dataset HIS1, HIS2. In other words a user can in this way for example predetermine a region of interest IB in a histopathology image dataset HIS1, HI S2 and the image processing algorithm TF-C automatically searches for all regions of similarity in the same histopathology image dataset HIS1, HIS2. A corresponding method is shown in FIG. 12. The sequence of the method steps is not restricted either by the sequence shown or by the numbering selected. Thus the sequence of the steps can be exchanged where necessary and individual steps can be left out.

A first step M10 is directed to the provision of histopathology image data. The histopathology image data can for example correspond to the first histopathology image data HIS1.

A second step M20 is directed to the provision of a region of interest IB. Step M20 in this case can be embodied like step S15′.

A third step M30 is directed to searching through the histopathology image data HIS1, HIS2 for regions of similarity AEB, with the regions of similarity AEB each having a similarity with the one or more regions of interest IB. The step of searching in particular features the application of (where necessary adapted) third image processing algorithm TF-C to the histopathology image data HIS1, HIS2. Apart from that step M30 can be embodied like step S30A′.

A fourth step M40 is directed to the provision of similarity information AEI based on the regions of similarity AEB. Step M40 in this case can be embodied similarly to step S30B′.

A fifth step M50 is directed to the provision of the similarity information AEI. In particular the provision of the similarity information AEI can comprise a display or highlighting of the regions of similarity in the histopathology image data HIS1, HIS2 for a user in a user interface 10.

The third image processing algorithm TF-C can, provided this has a trained function, be adapted thereby to the method according to FIG. 12 by training datasets being provided, which comprise a training region of interest IB and training histopathology image data HIS1, HIS2 as well as associated verified regions of similarity AEB in the training histopathology image data. The verified regions of similarity AEB in this case can again be based on an annotation of a user during an analysis or appraisal of the histopathology image data. Training can then comprise an input of the training region of interest IB and the training histopathology image data HIS1, HIS2 into the third image processing algorithm TF-C in order to create corresponding output data. These output values are then compared with the verified regions of similarity AEB. Based on the comparison the third image processing algorithm TF-C can then be adapted.

Where this has not happened explicitly, but is sensible and in the spirit of the invention, individual example embodiments, individual or their subaspects or features can be combined with one another or exchanged, without departing from the framework of embodiments of the current invention. Advantages of the invention described with regard to one example embodiment also apply, without this being explicitly stated, where they are able to be transferred, to other example embodiments.

The following points are likewise part of the disclosure.

1. A computer-implemented method for provision of similarity information (AEI) regarding different histopathology image data (HIS1, HIS2) of a patient with the steps:

provision (S10′, S10″) of first histopathology image data (HIS1), which is based on a tissue sample that was taken from a patient at a first point in time;

provision (S20′, S20″) of second histopathology image data (HIS2), which is based on a tissue sample that was taken from the patient at a second point in time different from the first;

identification (S15′, S15″) of a region of interest (IB) in the first histopathology image data (HIS1);

searching (S30A′, S30A″) the second histopathology image data (HIS2) for regions of similarity (AEB), with the regions of similarity (AEB) each having a similarity with the region of interest (IB), wherein the step of searching (S15′, S15″) comprises the application of an image processing algorithm (TF-A, TF-B, TF-C, TF-A′) to the second histopathology image data (HIS2);

determination (S30B′, S30B″) of similarity information (AEI) based on the step of searching (S30A′, S30A″); and provision (S40′, S40″) of the similarity information (AEI).

2. The method according to point 1, in which

the region of interest (IB) has one or more individual regions (ROI, ROI1, ROI2, ROI3) defined in the first histopathology image data (HIS1), which in particular each indicate a pathological appraisal.

3. The method according to one of the preceding points, in which the similarity information (AEI) comprises:

a specification of the regions of similarity (AEB);

a quantitative specification of the respective similarity of the regions of similarity (AEB);

an item of location information of the regions of similarity (AEB) in the second histopathology image data (HIS2);

an assistance image (AB) based on the second histopathology image data (HIS2), in which the regions of similarity (AEB) are highlighted;

a probability that a recidive relationship exists between the first and second histopathology image data (HIS1, HIS2).

4. The method according to one of the preceding points, in which

the first point in time lies before the second point in time.

5. The method according to point 4, in which

the step of provision of the first histopathology image data (HIS1) comprises:

access to a database (60) for histopathology image data;

selection of the second histopathology image data (HIS2) from the histopathology image data stored in the database (60) based on the first histopathology image data (HIS1) and/or metadata assigned to the first histopathology image data (HIS1).

6. The method according to point 5, in which the metadata has:

a patient identifier for identification of the patient,

information about an anatomical region of the patient from which the tissue sample was taken, on which the first histopathology image data (HIS1) is based, and/or

information regarding the first point in time.

7. The method according to one of the preceding points, in which

the second point in time lies before the first point in time.

8. The method according to point 7, in which

the step of provision (S20′) of the second histopathology image data (HIS2) comprises:

access to a database (60) for histopathology image data;

selection of the second histopathology image data (HIS2) from the histopathology image data stored in the database (60) based on the first histopathology image data (HIS1) and/or metadata assigned to the first histopathology image data (HIS1).

9. The method according to point 8, in which

the metadata has:

a patient identifier for identification of the patient,

information about an anatomical region of the patient from which the tissue sample was taken, on which the first histopathology image data (HIS1) is based,

information in respect of the histopathological staining used in the provision of the first histopathology image data (HIS1) and/or

information regarding the first point in time.

Even if not explicitly stated, individual example embodiments, or individual sub-aspects or features of these example embodiments, can be combined with, or substituted for, one other, if this is practical and within the meaning of the invention, without departing from the present invention. Without being stated explicitly, advantages of the invention that are described with reference to one example embodiment also apply to other example embodiments, where transferable.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for provision of similarity information regarding different histopathology image data of a patient, the method comprising:
provisioning first histopathology image data, based on a tissue sample taken from a patient at a first point in time;
provisioning second histopathology image data, based on a tissue sample taken from the patient at a second point in time, different from the first point in time;
analyzing, using an image processing algorithm, the first histopathology image data and the second histopathology image data for a similarity between at least one region from the first histopathology image data indicating a pathological appraisal and at least one region from the second histopathology image data indicating a pathological appraisal;
determining similarity information based on the analyzing of the image processing algorithm for the similarity; and
provisioning the similarity information determined.

2. The method of claim 1, wherein the similarity information includes at least one of:
a specification of similar regions in at least one of respective first histopathology image data and second histopathology image data indicating a pathological appraisal;
a quantitative specification of respective similarity of similar regions in at least one of respective first histopathology image data and second histopathology image data indicating a pathological appraisal;
location information of similar regions in at least one of the first histopathology image data and second histopathology image data indicating a pathological appraisal;
an assistance image based on histopathology image data the first histopathology image data and second histopathology image data, in which similar regions indicating a pathological appraisal are highlighted; and
a probability that a recidive relationship exists between the at least one of the first histopathology image data and second histopathology image data.

3. The method of claim 1, wherein the provisioning of the similarity information features a display of the similarity information for a user via a user interface.

4. The method of claim 1, further comprising:
filling out a medical report template based on the similarity information.

5. The method of claim 1, wherein the determining of the similarity information comprises:
identifying a region of interest in the first histopathology image data indicating a pathological appraisal;
searching through the second histopathology image data for regions of similarity, with the regions of similarity each having a similarity with the region of interest, wherein the searching includes application of the image processing algorithm to the second histopathology image data; and
determining the similarity information based on the searching.

6. The method of claim 5, wherein the region of interest has one or more individual regions defined in the first histopathology image data.

7. The method of claim 5, wherein the identifying of the region of interest comprises at least one of:
determining the region of interest by the image processing algorithm, and
evaluating an annotation of a user, the annotation characterizing the region of interest.

8. The method of claim 5, wherein the searching comprises:
extracting a feature signature based on the region of interest; and
establishing the similarity information based on the feature signature extracted.

9. The method of claim 1, wherein the image processing algorithm includes a trained function.

10. The method of claim 9, wherein the trained function includes a convolutional neural network.

11. The method of claim 1, wherein the second point in time lies before the first point in time.

12. The method of claim 11, wherein the provisioning of the second histopathology image data comprises:
accessing a database for histopathology image data; and
selecting the second histopathology image data from the histopathology image data stored in the database based on at least one of the first histopathology image data and metadata assigned to the first histopathology image data.

13. The method of claim 12, wherein the metadata includes at least one of:
a patient identifier for identification of the patient,
information about an anatomical removal region of the patient from which the tissue sample was taken, on which the first histopathology image data is based,
information in respect of the histopathological staining used in the provisioning of the first histopathology image data, and
information about the first point in time.

14. A system for provision of similarity information regarding different histopathology image data of a patient, comprising:
an interface embodied to receive first histopathology image data and second histopathology image data, the first histopathology image data being based on a tissue sample taken from a patient at a first point in time, and the second histopathology image data being based on a tissue sample taken from the patient at a second point in time, different from the first; and
a computing device embodied to:
determine, based on the first histopathology image data and second histopathology image data, similarity information using an image processing algorithm, the similarity information including a specification of similarity between at least one region from the first histopathology image data indicating a pathological appraisal and at least one region from the second histopathology image data indicating a pathological appraisal; and
provide the similarity information determined.

15. The system of claim 14, further comprising:
a database to store a number of items of histopathology image data; and
a user interface for interaction with a user, the interface including a data connection to the database and the user interface, and
wherein the computing device is further embodied to:
select the first histopathology image data from the database based on a manual input by the user, into the user interface, and receive the first histopathology image data via the interface, and
select the second histopathology image data from the database based on at least one of the first histopathology image data and metadata assigned to the first histopathology image data.

16. A non-transitory computer program product, storing a program, directly loadable into a memory of a programmable computing device a controller, including program code for carrying out the method of claim 1 when the program is executed in the controller.

17. A non-transitory computer-readable memory medium, storing readable and executable program sections for executing the method of claim 1 when the program sections are executed by a controller.

18. The method of claim 2, wherein the provisioning of the similarity information features a display of the similarity information for a user via a user interface.

19. The method of claim 2, further comprising:
filling out a medical report template based on the similarity information.

20. The method of claim 18, further comprising:
filling out a medical report template based on the similarity information.

21. The method of claim 2, wherein the determining of the similarity information comprises:
identifying a region of interest in the first histopathology image data indicating a pathological appraisal;
searching through the second histopathology image data for regions of similarity, with the regions of similarity each having a similarity with the region of interest, wherein the searching includes application of the image processing algorithm to the second histopathology image data; and
determining the similarity information based on the searching.

22. The method of claim 6, wherein the one or more individual regions defined in the first histopathology image data each indicate a pathological appraisal.

23. The method of claim 7, wherein the annotation is provided by a manual input of a user via a user interface after the provisioning of the first histopathology image data.

24. The method of claim 10, wherein the trained function includes a region-based convolutional neural network.
